(19) United States
(12) Patent Application Publication
(10) Pub. No.: US 2024/0156444 A1
(43) Pub. Date: May 16, 2024

(54) AUTOMATION COMPATIBLE COLLECTION DEVICE FOR BIOLOGICAL SAMPLES

(71) Applicant: Rhinostics Inc., Waltham, MA (US)

(72) Inventors: Cheri Walker, Wayland, MA (US); Richard Novak, Waltham, MA (US); Anna Simpson, Waltham, MA (US); Christopher Negus, Waltham, MA (US)

(21) Appl. No.: 18/493,030

(22) Filed: Oct. 24, 2023

Related U.S. Application Data
(60) Provisional application No. 63/418,869, filed on Oct. 24, 2022.

(51) Int. Cl. A61B 10/00; A61B 10/02

(52) U.S. Cl. CPC A61B 10/0096; A61B 10/0233

(57) ABSTRACT

A collection device can include a cap configured to interface with automation equipment used in a testing facility (e.g., a handheld or fully automated decapping machine, robotic decappers, handheld decappers, liquid handlers, transport robotics, shaking apparatus, etc.) for automated processing (e.g., decapping, rapid accessioning, sample testing, or other processing steps). Following collection of a sample, the sample can be inserted or screwed into a vial, the cap can be coupled to the vial, and the vial can be transported to an automation system or devices to perform automated processing (e.g. decapping, sample testing, or other processing steps). Thus, such collection devices may provide a convenient, efficient, and cost-effective apparatus, system and method for transporting and automated testing of samples.

### CROSS-REFERENCE TO RELATED APPLICATIONS

This application claims priority to and the benefit of U.S. Provisional Application No. 63/418,869, filed Oct. 24, 2022, of which is incorporated herein in its entirety.

### FIELD OF THE INVENTION

The present disclosure generally relates to collection devices used for collection of samples and, more particularly, to automation compatible collection devices having features adapted for the improved collection of biological samples and/or interaction with automated equipment and related methods.

### BACKGROUND

The present disclosure generally relates to collection devices used for collection of samples and, more particularly, to automation compatible collection devices having features adapted for the improved collection of biological samples and/or interaction with automated equipment and related methods. Biological sample collection is an essential part of many testing methodologies including human and animal diagnostics, environmental testing, animal testing, applied life science testing, home and medical collection of human or animal biological samples, human identification and forensics, biobanking, sample storage and archiving, neonatal and other human screening tests, tracing and other important health, safety and other protocols and procedures.

The COVID-19 pandemic validated the home collection market for diagnostic testing and the need to remove bottlenecks from inefficient laboratory workflows for these tests. Blood and biological sample collection typically requires a phlebotomist or other medical personnel and can be painful or intimidating and typically must be done through a visit to a medical clinic. Current paper-based collection devices are easy to collect but require them to be hole-punched in order to enter the diagnostic workflow, where they need manual or inadequately automated devices. Other solutions are too expensive and are not realistic for these cost-sensitive collection environments. Improvements that allow easy patient self-collection also aid medical personnel with easier and less painful sample collection in the clinical environment.

Other types of biological sample collection devices are rudimentary devices with few innovations, e.g., swabs having an ovular or bulbous shape at the end of stem or flat paper cards. While such conventional devices are often designed to make contact with various sample collection sites, they often do not collect and/or retain enough sample as desired or required for a particular testing application nor do they release the sample with efficiency for the desired testing. Additionally, they require largely inefficient and manual steps to bring the sample into the laboratory workflow using conventional devices. As another example, capillary blood collection has been used for decades to collect biological samples from patients. Capillary blood can be obtained by pricking a skin surface of the patient to cause a volume of blood to appear on the surface. The specimen can then be collected with a pipette, cuvette, absorbent paper, or capillary device. In some instances, the sample is then placed on a glass slide or a piece of filter paper for storage and/or further testing. In some cases, a medical specialist collects the sample but in a growing number of cases, the patient self-collects the sample and sends it for testing to a central location. In other cases, cellular samples such as buccal, pox, wounds, sores, melanoma, are being collected with devices/swabs that were not designed to collect or release material and are inefficient for the testing facilities that move the samples into assay workflows.

Clinical and medical laboratories are struggling with labor shortages, where turnover rates can be up to or higher than 25% and medical technologists are difficult to recruit and retain. With increasing labor costs and shortages, automating difficult sample collections for truly handsfree workflows can bring significant much-needed cost savings and reduction of labor dependency. Patient blood collection can be challenging and there are few formats available, the majority being an absorbent paper card that is collected by the medical professional or at home by the patient and then shipped to the testing location. Because these materials are difficult to automate, requiring a hole-punch step to remove the sample from a flat card material, these formats are time consuming and challenging for the testing facility to handle as they move the sample into the assay modality.

Absorbent paper dried blood cards (DBCs) have large circles that can be hard to fill completely, risking a less concentrated sample taken via hole punch for processing at the lab. In the United States and many countries around the world, newborns are subjected to a heel-prick lancet where blood is collected for a panel of genetic tests using DBCs. These samples require rapid turnaround times and create a significant amount of testing volume that would benefit from the ability to automate how these samples are brought into the laboratory assay workflow. The easy collection and automation solution combination provided by the devices in this application will significantly improve this collection and testing process. Human identity and forensic testing are used on a global basis to collect, test, and catalogue human samples related to crime, disaster recovery, unknown person identification, and other similar applications.

There exists significant backlog for testing such samples because of the poor automation systems that exist for these workflows. Easy blood and other biological sample collection combined with automation-enabled devices can significantly improve the workflows and remove the backlogs. One primary method of collection for DNA testing includes using a standard swab inside the cheek to collect buccal cells that can be extracted to release DNA for genetic testing of a wide variety of applications. Currently, these swabs are hand-decapped and side accessioned into the workflows. Bring an automated buccal collection device would allow for handsfree workflows that remove time, manual steps, and labor costs.

In addition, transferring a sample to an assay device using conventional collection techniques can be challenging and time consuming. There is also not always compatibility between commercially available collection devices and the testing device used to assay the samples. Thus, there is a need for improved sample collection devices that better retains samples and/or integrate with testing equipment (e.g., automated testing equipment) and where the processes can be automated for handsfree, robotic workflows that bring efficiencies to the testing facilities.

### SUMMARY

Accordingly, the present disclosure relates to automation compatible, purpose-built collection devices with features adaptable to collect samples such as for example DNA-RNA-protein, cellular, buccal, tissue, wound, pox, tissue, fecal, melanoma and other dermatological samples, and fluids samples (e.g., blood or bodily fluids in humans or animals). In some embodiments, the automation compatible collection devices can enable easy transport to a testing facility. In some embodiments they include easy, ergonomic collection by the patient or physician and system in addition to simplify preparation and shipment to the laboratory or other location for testing, banking or storage.

As described herein, the samples for life sciences, research, clinical trials, applied markets, diagnostics, screening tests, human identity, home healthcare and testing applications, toxicology, drug testing, contract research, animal testing, veterinary testing, identity, ancestry, wellness testing, food sensitivity testing, biomarker testing, forensics, clinical trials, environmental, water, food, and soil collection, and/or other types of tests (e.g., antigen testing, hormone level testing, genetic testing, vaccination status, antibody level testing, blood smears, complete blood count (CBC), hemoglobin, hematocrit, electrolyte panel, neonatal blood gasses, neonatal bilirubin, neonatal screening, glucose, lipids (cholesterol, HDL cholesterol, LDL cholesterol), cancer diagnostics, nucleic acids (DNA/RNA) including but not limited to microarrays, polymerase chain reaction (PCR), next generation sequencing; A1C levels, cancer and other biomarkers, blood glucose levels, triglycerides, infectious diseases, pathogens, pharmacology, sexually transmitted diseases (HIV, HCV, HBV, syphilis, and others), other disease state testing, drug testing, chemical testing, and/or all other blood and biological sample tests) can be collected from any biological anatomy suitable for collection and suitable applied or environmental collection locations. For example, the samples can be blood, biological fluids, cellular samples, DNA samples, RNA samples, protein samples, buccal cells and any other cell samples, wound samples, tumor samples, melanoma and dermatological, pox and other sores samples, tissue samples, blood samples, saliva samples, mucus samples, and/or other biological fluids samples or biological tissues (e.g. buccal cells, sores, poxes, tissues, tumors or pathogens). In addition, urine can be collected directly from the urine stream or from a collection device such as a collection cup and used for a wide variety of life science research, animal testing, toxicology, screening tests, drug testing, diagnostic and other broad urine testing applications. Samples can come from the environment, including but not limited to soil, water, facility testing. The samples in the present disclosure can be collected using a capillary (e.g., a complete tube capillary, a three-sided capillary, a parallel structure capillary or other suitable capillaries) and/or other structures or devices capable of collecting and retaining, drying and/or transferring samples as needed. The samples can also be collected on an absorbent paper or other material. Additionally, the device can include structures for physical collection, transfer or retention of a wide variety of biological and environmental samples.

In general, the collected samples can be used to perform any suitable assay or test, e.g., antigen testing, hormone level testing, genetic testing, tests associated with vaccination status, antibody level testing, blood smears, complete blood count (CBC), hemoglobin, hematocrit, electrolyte panel, neonatal blood gasses, neonatal bilirubin, neonatal screening, glucose, lipids (cholesterol, HDL cholesterol, LDL cholesterol), cancer diagnostics, nucleic acids (DNA/RNA) including but not limited to microarrays, polymerase chain reaction (PCR), next generation sequencing; A1C levels, cancer and other biomarkers, blood glucose levels, triglycerides, infectious diseases, pathogens, pharmacology, sexually transmitted diseases (HIV, HCV, HBV, HAV, HSV, syphilis and other), other disease state testing, drug testing, chemical testing, and/or all other blood and biological sample, pathogens, cancer testing, genetic testing, food sensitivity, wellness testing, toxicology, and/or all other blood tests or other sample collections. Various embodiments of the collection devices as described herein include a sample collection head or in some cases, a capillary and/or a cap.

Advantageously, the collection device can include a cap configured to interface with automation equipment used in a testing facility (e.g., a handheld or fully automated decapping machine, robotic decappers, handheld decappers, liquid handlers, transport robotics, shaking apparatus, etc.) for automated processing (e.g., decapping, rapid accessioning, sample testing, or other processing steps). Following collection of a sample, the sample can be inserted or screwed into a vial, the cap can be coupled to the vial, and the vial can be transported to an automation system or devices to perform automated processing (e.g. decapping, sample testing, or other processing steps). Thus, such collection devices may provide a convenient, efficient, and cost-effective apparatus, system and method for transporting and automated testing of samples.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIGS. 1A-1E are images of an exemplary automation compatible collection device including a complete capillary, according to various embodiments;

FIGS. 2A-2B are images of various exemplary automation compatible collection devices including incomplete capillaries with various structures, according to various embodiments;

FIGS. 3A-3C are images of an exemplary automation compatible collection device including an incomplete capillary structure, according to various embodiments;

FIGS. 4A-4D are images of another exemplary automation compatible collection device including an incomplete capillary structure, according to various embodiments;

FIGS. 5A-5D are images of yet another exemplary automation compatible collection device including an incomplete capillary structure, according to various embodiments;

FIGS. 6A-6C are images of yet another exemplary automation compatible collection device including an incomplete capillary structure, according to various embodiments;

FIGS. 7-14 are schematic views of various exemplary automation compatible collection devices, according to various embodiments;

FIGS. 15-24 are schematic, detail views of various additional features of exemplary automation compatible collection devices, according to various embodiments;

FIG. 25 shows schematic perspective views of an exemplary automation compatible collection device manufactured by injection molding, according to various embodiments;

FIGS. 26-33 are schematic, detail views of various exemplary sample collection heads of automation compatible collection devices for collecting biological samples, according to various embodiments;

FIG. 34 shows images of exemplary materials for manufacturing a collection device, according to various embodiments;

FIG. 35 is an image of an exemplary automation compatible collection device including an incomplete capillary structure, according to various embodiments;

FIGS. 36A and 36B are images of an exemplary 96-well rack that can be used with vials containing samples using a collection device, according to various embodiments;

FIG. 37A is an image of an automated decapper;

FIG. 37B is an illustration of a rack of an automated decapper;

FIG. 38 illustrates a rack 3800 with a 24 well rack format;

FIGS. 39A-D illustrates a collection device with an absorbent insert;

FIG. 40 is another image of the collection device;

FIGS. 41A-E show various formats of a collection device with an absorbent insert;

FIGS. 42A-B are images of collection devices with absorbent inserts;

FIG. 43 is an image of various collection devices in vials;

FIGS. 44A-D illustrates a collection device with capillaries;

FIG. 45 is an image of a collection device in a universal size and/or large format;

FIG. 46 illustrates a collection device with a high volume capillary;

FIG. 47 illustrates a collection device with a collection tip;

FIGS. 48A-B are images of a collection device with a collection tip;

FIGS. 49A-B illustrates a collection device with and without a vial;

FIGS. 50A-B illustrate molds for a collection device; and

FIG. 51 illustrates various compression molding of powders.

### DETAILED DESCRIPTION

Various embodiments of the present invention are directed to automation compatible collection devices that are better adapted to collect samples than prior collection devices. Various components and features of the collection device, according to various embodiments, are described in greater detail below. This application will often describe the technology as directed with an ergonomic, easy collection device using the cap combined with the sample collection portion of the device. This application will often describe the technology as directed to an automation compatible collection device.

In some embodiments, the collection device in the present disclosure includes a capillary (complete or incomplete) and/or an automation compatible cap attached to the capillary. As used herein, the term “complete capillary” refers to a capillary that forms a fully enclosed lumen, i.e., the cross-sectional shape of the capillary forms an enclosed shape. As used herein, the term “incomplete capillary” refers to a capillary that forms a non-enclosed lumen, i.e., the cross-sectional shape of the capillary forms a non-enclosed shape. In some embodiments, the device contains an immobilized absorbent paper or other material. In some embodiments, the inventions described herein can apply to any type of collection device, capillary or other types of devices, with or without a cap. For example, in various embodiments, the inventions can be applied to swabs, lancets, cuvettes, novel formats to collect and retain cells and tissues, and other known devices for collecting samples.

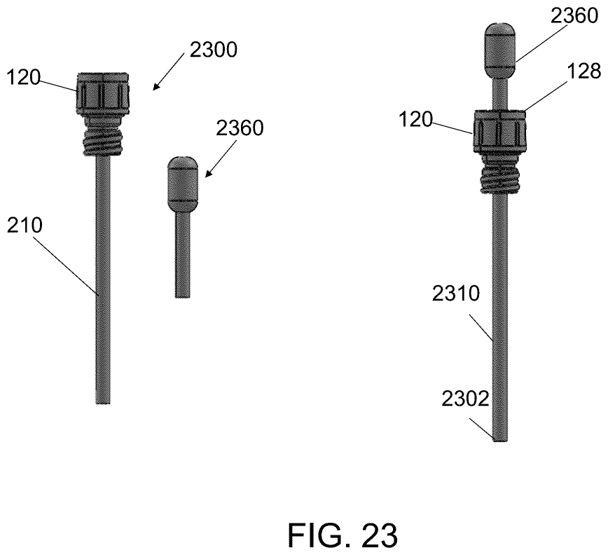

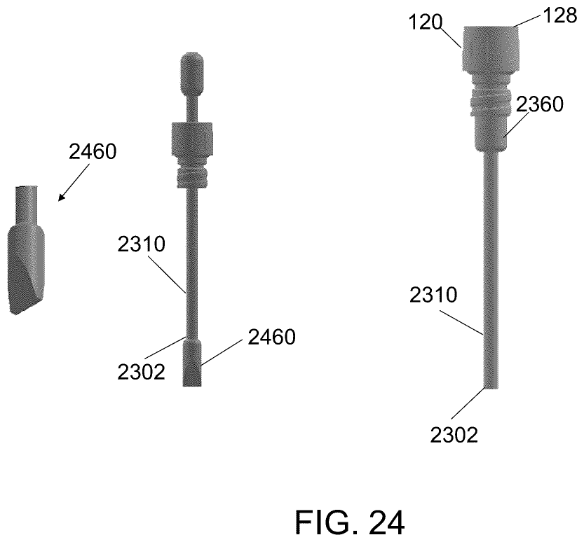

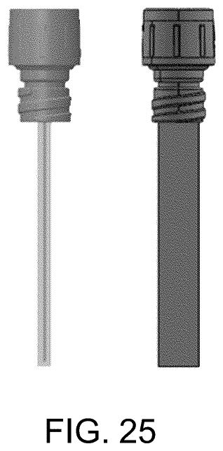

In some embodiments, the collection device includes other sample (e.g. blood, cells, tissues) collection structures. In some embodiments, the collection device in the present disclosure includes mechanical devices, such as a suction device (FIG. 23 or 24), a duck-bill valve (FIG. 24), and/or other suitable devices to obtain fluids. For example, the mechanical device can generate a pressure gradient to pull or draw fluids using e.g., suction, pumping, and/or vacuum mechanisms.

Collection Device

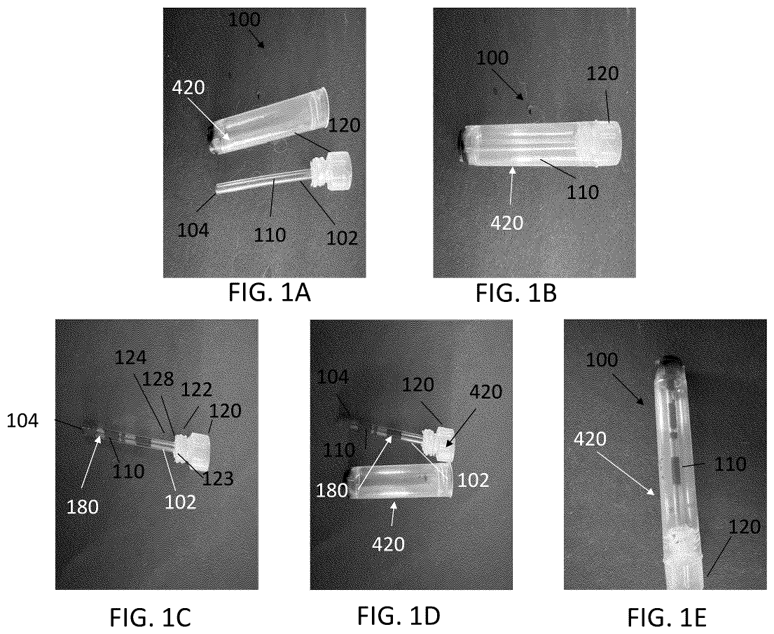

In general, the collection device as described herein (e.g., collection devices 100, 200, 300, 400, 500, or 600 as shown in FIGS. 1-6) can be used to collect any suitable biological fluids (e.g., blood from a finger or vein, urine, weeping sores, tears, pus, wound debris) at any biological location (e.g., finger, arms, legs, heel, or other suitable regions) of a subject (e.g., a human, an animal, etc.) or surface inside a facility or in the environment. The collection device as described herein can be also used to collect any biological tissues (e.g. buccal cells, sores, poxes, tissues, skin, wound materials, tumors or pathogens).

In some embodiments, the collection device can be used to collect a sample for research, tests, clinical trials, environmental testing, food testing, forensics, biobanking, sample storage, and other applied markets and/or any other uses from sample collections. In some embodiments, the collection device can be used to perform one or more tests (e.g., for analysis and/or detection) as described above on a subject (human or animal).

In other embodiments, the sample includes cells collected from a variety of locations including but not limited to the inside of the cheek, mouth, vagina, penis, anus, skin, cells, wounds, poxes, sores, tissues, tumors, dermatological collection including melanoma.

In some embodiments, the collection device can be used to collect a sample for determination of physiological and/or biochemical states, such as disease, mineral content, toxicology, pharmaceutical drug effectiveness, and organ function.

In some embodiments, the collection device can be used to collect a sample from a subject infected with or suspected to be infected with a sexually transmitted disease (STD). In some embodiments, the STDs include at least one of chlamydia, genital herpes, genital warts or human papillomavirus, gonorrhea, hepatitis A, hepatitis B, hepatitis C, syphilis, trichomoniasis, human immunodeficiency virus (HIV), cytomegalovirus, molluscum contagiosum, Mycoplasma genitalium, bacterial vaginosis, or others.

In some embodiments, the collection device can be used to collect a sample from a subject for genetic testing or screening tests. In some embodiments, the subject is being tested to examine genetic status such as hereditary cancer, pharmacogenetic status, neonatal and other genetics panels, any disease risk, wellness testing, food sensitivity, recessive or dominant gene risk for offspring, ancestry, identity, forensics, or any other genetic test. In some embodiments, the testing measures RNA levels transcribed from specific genes. In some embodiments, the genetic testing can be done by polymerase chain reaction, SNPs, microarrays, next generation sequencing, mass spectrometry, Sanger sequencing, and all other assay modalities that measure DNA and RNA levels.

In some embodiments, the collection device can be used to collect a sample from a subject infected with or suspected to be infected with an infection detectible in blood of a subject. In some embodiments, the subject is infected with or suspected to be infected with an infection, in other cased the following levels can be measured such as hormone levels, A1c and blood glucose levels (e.g., for monitoring or detection of diabetes), antibody levels (e.g., for analysis of immune status), presence of cancer or tumor markers, or antigen levels. In some embodiments, the subject is infected with or suspected to be infected with tuberculosis (TB).

In some embodiments, the collection device is adapted to collect toxicology samples for toxicology (e.g., toxicological test or screening).

In some embodiments, the collection device is adapted to collect environmental samples, such as surface areas, water and/or soil, for environmental testing.

In some embodiments, the collection device is adapted to collect biological samples for surveillance (e.g., surveillance of contamination of facilities).

In some embodiments, the collection device is adapted to collect forensic samples (e.g., blood) on site of crime or from victims of crime for forensic testing. In some embodiments, the collection device may be adapted for determining human identification. The collection device may bank or otherwise store samples that may be used for determining human identification. In some embodiments, the collection device may be adapted to measure cancer, tumor markers, and the like in blood or other tissues, such as, but not limited to, skin.

In some embodiments, the collection device is adapted to collect samples, such as biological samples from animals for animal studies, veterinary testing, animal husbandry and preclinical trial testing of animals.

In some embodiments, the collection device is automation compatible.

In some embodiments, the collection device, vial, and/or their components (e.g., capillary, cap, and any portions of these) are disposable, reusable, biodegradable, compostable, and/or recyclable.

In some embodiments, the collection device, vial, and/or their components (e.g., capillary, cap, and any portions of these) are configured to include a smooth surface without abrasive or sharp features, in order to ensure safe use of the collection device and to avoid damage to the subject or injury to the sample collection operator.

In some embodiments, changes to the surface are achieved by surface treatments and/or treatment with chemicals (e.g., cellulose acetate, heparin, or other suitable chemicals).

In some embodiments, at least part of the surface treatment can allow separation of blood or other fluid samples into different components including but not limited to plasma or serum

In some embodiments, the surface treatment includes cross hatching, oxidation, mechanical abrasion, mechanical machining, mechanical texturing, chemical reaction, chemical etching, plasma or other high energy reactions, surface coating with one or more materials homogeneously or heterogeneously, and/or other suitable treatment methods.

In some embodiments, the surface treatment changes the surface properties of the collection device, such as the hydrophilicity, chemical reactivity, and/or biological reactivity (e.g., coagulation) of the collection device.

In some embodiments, the collection device, vial, and/or their components (e.g., capillary, cap, and any portions of these) includes features or elements (e.g., desiccation, anticoagulants, or other suitable elements) adjust the speed at which the collected sample fluid dries or coagulates in the collection device.

In some embodiments, the collection device, vial, and/or their components (e.g., capillary, cap, stem, sample collection head, and/or any portions of these) are compatible with standard wells for 1536-well plate formats, 384-well plate formats, 96-well plate formats, 48-well plate formats, 36-well plate formats, 24-well plate formats, 12-well plates formats, or any other standard well-plate formats. In some embodiments, the collection device, vial, and/or their components (e.g., capillary, cap, stem, sample collection head, and/or any portions of these) are compatible with universal, large format wells for 48-well plate formats, 36-well plate formats, 24-well plate formats, 12-well plates formats, or any other standard well-plate formats. For larger tube processing, for example 12-14 mm diameter tubes.

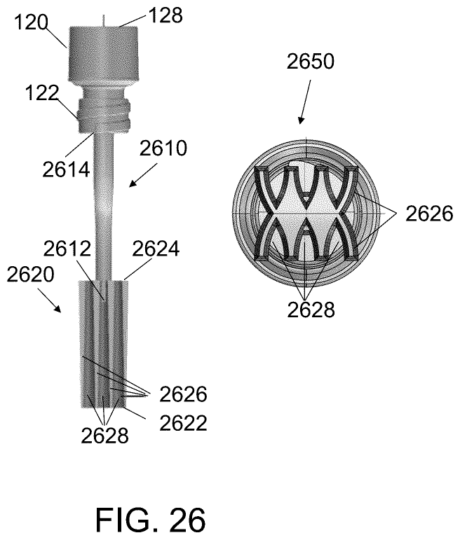

In general, the sample collection device in the present disclosure as described herein can take any known form. For example, the sample collection device can include at least one complete capillary, incomplete capillary, stem, sample collection head, or their combinations thereof, which is coupled to or separate from other elements such as a cap for the collection of samples, as described below in further detail. In another example, the sample collection does not include a capillary (e.g., FIGS. 26-33). For instance, the capillary can be replaced by a stem and/or a sample collection head, where the sample collection head can include any structures, such as fins, flat surfaces, nubs, bars, cavities, combinations thereof, and/or any other porous or non-porous structures to collect biological samples. In particular embodiments, the sample collection device includes any one of an absorbent paper, fibers, foams, or other materials or structures. In particular embodiments, the sample collection device can include any one of an absorbent paper, fibers, foams, or other materials or structures attached to an automation compatible cap.

In some embodiments, the biological samples include blood, tears, urine, cells, DNA, RNA, protein, cellular, wound, pox, tissue, and/or any other biological samples. In some embodiments, the biological samples are collected from a collection area such as skin, and/or a portion of biological materials such as blood, vaginal fluids, anal fluids, wound tissues, pus, semen, saliva, urine, feces, hair, teeth, bone, tissue and/or cells.

In some embodiments, the capillary can include any configuration that is sufficient to collect a sample from a subject.

In some embodiments, the capillary includes one or more complete capillaries, one or more incomplete capillaries, or their combinations thereby. For example, in some embodiments, the collection device includes an incomplete capillary at the proximal end, and a complete capillary at the distal end. In another example, in some embodiments, the collection device includes an incomplete capillary at the distal end, and a complete capillary at the proximal end. In another example, the capillary includes one or more capillaries disposed within a larger capillary.

In some embodiments, at least part of the capillary is of an absorbent head type. For example, the capillary can be flocked, foam, spun fibers, etc. In some embodiments, the capillary is coated by at least one absorbent material (e.g., flocked materials, plant-derived materials, animal-derived materials, synthetic materials, foams, a combination of these, or other suitable materials). In some embodiments, the capillary is non-absorbent (e.g., not flocked, not coated by fibrous materials). In some embodiments, the capillary, collection tube, or other devices described may be coated in ethylenediaminetetraacetic acid (ETDA), heparin, boric acid, and/or other chemicals or compounds.

In some embodiments, the collection device includes a porous collection zone (e.g., channel or interior of a capillary).

In some embodiments, the collection device includes a non-porous collection zone (e.g., channel or interior of a capillary).

In some embodiments, mechanical methods (e.g., methods using suction or pressure differences) are used to collect, retain, or transfer fluids.

Complete Capillary

FIGS. 1A-1E are images of an exemplary collection device 100, according to various embodiments. FIGS. 1A and 1B are perspective views of the collection device 100 and an exemplary vial 420 for receiving the collection device 100 e.g., before collecting biological fluids or biological tissues. FIGS. 1C-1E are perspective views of the collection device 100 and the vial 420 including collected biological fluids (e.g., blood 180) and/or biological tissues (e.g. buccal cells, sores, poxes, tissues, tumors or pathogens).

In some embodiments, at least part of the capillary 110 is a complete capillary such as a pipette or a capillary tube.

In some embodiments, the collection device (e.g., collection device 100 or 200) in the present disclosure includes a capillary and/or a cap. In some embodiments, the collection device in the present disclosure includes 2, 3, 4, 5, or more capillaries, caps, absorbent papers, fibers, foams, stems, sample collection heads, and/or other materials or structures. In some embodiments, the capillary 110 is separate from or not coupled to a cap 120. In some embodiments, the cap 110 is operatively, removably, and/or firmly coupled (e.g., attached) to the capillary 110. In some embodiments, the cap is irremovable and locked with the capillary 110. In some embodiments, the capillary 110 is replaced by any other structures such as a stem (e.g., stem 2610 in FIG. 26) and/or a sample collection head.

In some embodiments, the cap 120 can be adapted to be operatively coupled to any collection device (e.g., blood collection device, biological sample collection device, swabs, and/or other sample collection devices from any part of a biological location of a subject).

In some embodiments, the capillary 110 can include an axial shaft (e.g., a hollow cylindrical rod). In some embodiments, the capillary 110 includes a proximal end 102 and a distal end 104. In some embodiments, the proximal end 102 and/or the distal end 104 include any suitable shape, e.g., rounded, pointed, grooved, and/or other suitable shapes.

In some embodiments, the capillary 110 and the cap 120 can be adapted to accommodate transport in a vial or a tube (e.g., a threaded tube) for securely storing and/or transport of collected samples, where the vial or tube has a width or diameter ranging from about 1 mm to about 20 mm, from about 5 mm to about 15 mm, from about 5 mm to about 15 mm, from about 12 mm to about 14 mm, or from about 6 mm to about 10 mm, and/or a height or length from about 10 mm to about 200 mm, from about 40 mm to about 100 mm, from about 40 mm to about 60 mm, from about 60 mm to 100 mm, or from about 65 mm to about 100 mm.

In some embodiments, the capillary 110 is configured to be open at one or both ends (e.g., at the proximal end 102 and/or the distal end 104) of the capillary 110. Such configuration can enable collecting samples from a sample collection location (e.g., vessels or capillary beds at fingertips or other parts of the body) and/or connecting other devices (e.g., a cap, lancet, needle, intravenous line or PICC line, tube, additional capillary, a finger-stick capillary bed, an automated device, or other suitable devices or elements).

In some embodiments, the capillary 110 includes a length (e.g., distance between the first proximal end 102 and the distal end 104) in a range of about 5 mm to about 200 mm, about 10 mm to about 175 mm, about 15 mm to about 420 mm, about 20 mm to about 125 mm, about 25 mm to about 100 mm, about 30 mm to about 75 mm, or about 35 mm to about 50 mm. For example, the capillary 110 can include a length of about any of: 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 110 mm, 120 mm, 130 mm, 140 mm, 420 mm, 160 mm, 170 mm, 180 mm, 190 mm, or 200 mm.

In some embodiments, the capillary 110 includes an outer diameter compatible with standard wells for 1536-well plate formats, 384-well plate formats, 96-well plate formats, 48-well plate formats, 36-well plate formats, 24-well plate formats, 12-well plates formats, or any other standard well-plate formats.

In some embodiments, the capillary 110 includes an outer diameter in a range from about 0.1 mm to about 6 mm, about 0.5 mm to about 5.5 mm, about 1.0 mm to about 5 mm, about 1.5 mm to about 4.5 mm, about 2 mm to about 4 mm, about 2.5 mm to about 3.5 mm, about 1 mm to about 50 mm, about 5 mm to about 45 mm, about 10 mm to about 40 mm, about 15 mm to about 35 mm, about 20 mm to about 30 mm. In some embodiments, the capillary 110 includes an outer diameter of about any of: 0.1 mm, 0.5 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, 15 mm, 20 mm, 25 mm, 30 mm, 35 mm, 40 mm, 45 mm, or 50 mm. In some embodiments, the capillary 110 includes an outer diameter of less than about any of: 0.1 mm, 0.5 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, 15 mm, 20 mm, 25 mm, 30 mm, 35 mm, 40 mm, 45 mm, or 50 mm.

In some embodiments, the capillary 110 includes the same outer diameter for the entirety of the capillary 110. In various embodiments, the capillary 110 includes at least two different outer diameters for the entirety of the capillary 110. For example, the diameter of a first portion (e.g., the proximal end 102) of the capillary 110 may be different from the diameter of a second portion (e.g., the distal end 104) of the capillary 110.

In some embodiments, the capillary 110 is a complete capillary and includes a hollow interior that is surrounded (e.g., fully surrounded as shown in FIG. 1) by the inner wall of the capillary 110. In some embodiments (e.g., FIG. 1), the inner wall of the capillary 110 defines an inner diameter (e.g., diameter of the hollow interior) of the capillary 110 in a range from about 0.1 mm to about 6 mm, about 0.5 mm to about 5.5 mm, about 1.0 mm to about 5 mm, about 1.5 mm to about 4.5 mm, about 2 mm to about 4 mm, about 2.5 mm to about 3.5 mm, about 1 mm to about 50 mm, about 5 mm to about 45 mm, about 10 mm to about 40 mm, about 15 mm to about 35 mm, about 20 mm to about 30 mm. In some embodiments, the hollow interior of the capillary 110 includes an outer diameter of about any of: 0.1 mm, 0.5 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, 15 mm, 20 mm, 25 mm, 30 mm, 35 mm, 40 mm, 45 mm, or 50 mm. In some embodiments, the capillary 110 includes an outer diameter of less than about any of: 0.1 mm, 0.5 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, 15 mm, 20 mm, 25 mm, 30 mm, 35 mm, 40 mm, 45 mm, or 50 mm.

In some embodiments, the capillary 110 includes the same inner diameter for the entirety of the capillary 110. In various embodiments, the capillary 110 includes at least two different inner diameters for the entirety of the capillary 110. For example, the diameter of a first portion of the hollow interior (e.g. at the proximal end 102) of the capillary 110 may be different from the diameter of a second portion of the hollow interior (e.g., at the distal end 104) of the capillary 110.

In some embodiments, the capillary 110, or at least a portion of the capillary 110 tapers (e.g., has sequentially reduced outer diameters and/or inner diameters) towards the proximal end 102, the distal end 104, from the middle of the capillary 110 or any point of the capillary 110. In some embodiments, the capillary 110 tapers from a maximum diameter at the proximal end 102 of the capillary 110 to a minimum diameter at the distal end 104 of the capillary 110.

In some embodiments, the capillary 110 tapers from a maximum diameter at the distal end 104 of the capillary 110 to a minimum diameter at the proximal end 102 of the capillary 110. In some embodiments, the maximum diameter of the capillary 110 occurs at any point of the capillary 110 and the diameters taper to a minimum diameter at the proximal end 102 and/or the distal end 104 of the capillary 110.

In some embodiments, the minimum diameter of the capillary 110 occurs at any point of the capillary 110 and the diameters taper to a maximum diameter at the proximal end 102 and/or the distal end 104 of the capillary 110.

In some embodiments, the diameter of the capillary 110 alternates between a minimum diameter and a maximum diameter from the proximal end 102 to the distal end 104 of the capillary 110.

In some embodiments, the capillary 110 (outer surface and/or hollow interior) can include a cross-section of at least one shape of a polygonal shape, e.g., a triangle, a square, a quadrilateral, a trapezoid, a pentagon, a hexagon, a polygon with at least 7, 8, 9, 10, 11, 12, 13, 14, 15, 16, 17, 18, 19, 20 or more sides, a circle, a circle with one or more flat sides, an ellipse, or an ellipse with one or more flat sides. In some embodiments, at least one side of the cross-section includes a convex and/or concave curve. In some embodiments, the cross-section includes a rotationally symmetric shape or an asymmetric shape. In some embodiments, the cross-section includes the same shape, or at least two different shapes for the entirety of the component. In some embodiments, texturing, grooves, or mechanical surface methods improve sample collection.

In some embodiments, the performance of the complete capillary (e.g., capillary 110) can be improved by adapting the length of the capillary, dimensions, and/or structures of the capillary.

Incomplete Capillary or Open Capillary

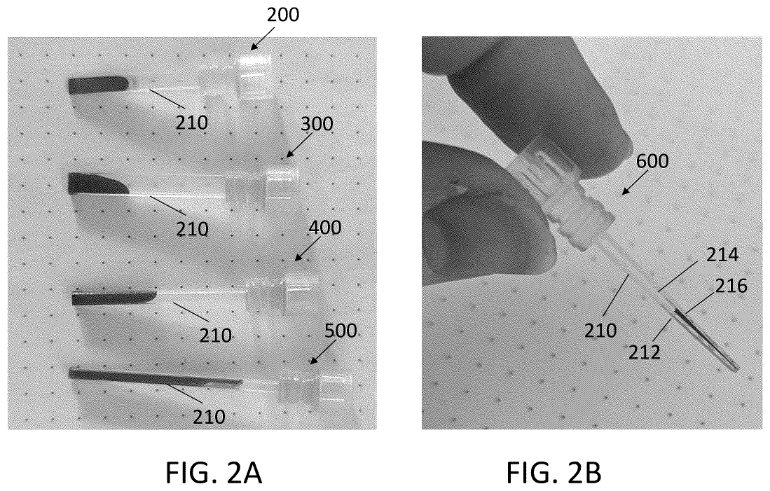

In some embodiments, the capillary is an incomplete capillary or open capillary (e.g., capillary 210), as shown in FIGS. 2-6 and 10-14.

Advantageously, an incomplete or open capillary as described herein can result in improved performance (e.g., in comparison with a closed or complete capillary), as described below.

In some embodiment, the open capillary can facilitate wicking of blood and/or drying of blood for transport, increase patient comfort, and/or safety.

In some embodiment, an open capillary can improve ease of use by increasing visibility of collected samples when collecting the samples. For example, the blood being collected in opaque capillaries can be visualized to a patient or physician. Accordingly, a desired volume of collected blood can be achieved.

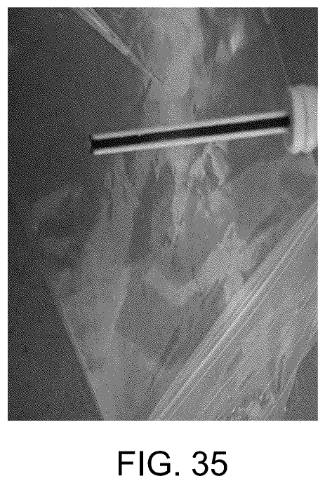

In some embodiment, an open capillary can result in easier and improved elution of samples. For example, such improve elution of blood can enable separation of blood into various components (e.g., red blood cell (RBC), white blood cell (WBC), and/or plasma), filter potential contaminants, and thus allowing optical tests. In another example, the blood can be collected through the entire length of capillary (e.g., FIG. 35), which can result in more rapid and complete sample elution and thus better assay performance.

In some embodiment, an open capillary can prevent bubbles during collection.

In some embodiment, an open capillary can provide or facilitate access to surface treatment of e.g., a molding surface or final part to modify surface properties.

In some embodiment, an open capillary can provide improved manufacturability. For example, it can enable injection mold of a single component as the final product, without the need to extrude a closed or complete capillary to assemble to a cap.

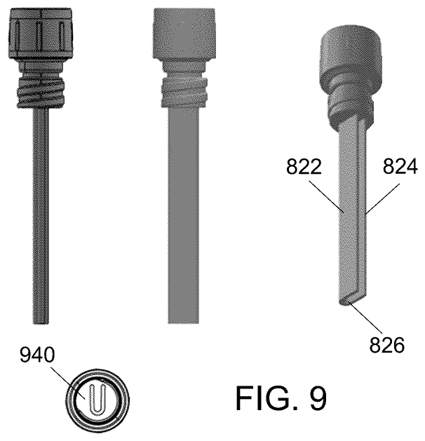

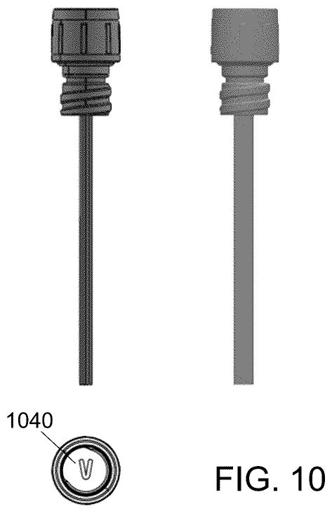

Thus, the sample collected by the incomplete capillary may be exposed to the environment (e.g., air). In some embodiments, the incomplete capillary includes a cross-section of one or a plurality of U-shapes (e.g., FIG. 2A, 4, 5, 6, or 9), V-shapes (FIG. 10), L-shapes (not shown), double L shapes (e.g., FIG. 2A, 2B, or 3), curved (e.g., semi-circle, truncated circle, semi-ellipse, truncated ellipse) shapes, polygonal shapes, or other suitable shapes with 1, 2, 3, or more sides or planes (e.g., flat or grooved sides or planes) (FIGS. 9, 11-14), or any combinations thereof.

In some embodiments, the cross-section of the incomplete capillary can be at least one of an incomplete circle, U-shape, V-shape, double L shape, curved shape, polygonal shape, or other suitable shapes or forms with about 5% to about 95%, about 5% to about 50%, about 10% to about 90%, about 15% to about 85%, about 20% to about 80%, about 25% to about 75%, about
30% to about 70%, about 35% to about 65%, about 40% to about 60%, about 45% to about 55%, of the circle or other shapes in closed forms missing).

In some embodiments, at least one side of the incomplete capillary is configured to include one or more convex curves, concave curves, gaps, holes, openings, or their combinations along the side of the capillary. Such configuration can increase exposure of the capillary to the environment and/or improve access for elution.

In some embodiments, the cross-section of the incomplete capillary includes a rotationally symmetric shape or an asymmetric shape.

In some embodiments, the cross-section of the incomplete capillary includes the same shape, or at least two different shapes for the entirety of the incomplete capillary. Grooves, texturing or other mechanical methods may improve sample collection and attraction/binding/release.

As shown in FIGS. 2-6, the collection devices 200, 300, 400, 500, or 600 includes a capillary 210 and/or a cap 120. In some embodiments, the capillary 210 is separate from or not coupled to a cap 120. In some embodiments, a cap 120 is operatively, removably, and/or firmly coupled (e.g., attached) to the capillary 210. In some embodiments, the cap 120 can be adapted to be operatively coupled to any collection device (e.g., blood collection device, swabs, and/or other sample collection devices from any part of a biological location of a subject).

In some embodiments, the capillary 210 includes a proximal end 202 and a distal end 204. In some embodiments, the proximal end 202 and/or the distal end 204 include any suitable shape, e.g., rounded, pointed, grooved, and/or other suitable shapes.

In some embodiments, the capillary 210 is configured to be open at one or both ends (e.g., at the proximal end 202 and/or the distal end 204) of the capillary 210. Such configuration can enable collecting samples from a sample collection location (e.g., vessels or capillary beds at finger tips or other parts of the body) and/or connecting other devices (e.g., a cap, lancet, needle, intravenous line or PICC line, tube, additional capillary, a finger-stick capillary bed, an automated device, or other suitable devices or elements).

In some embodiments, the capillary 210 includes a length (e.g., distance between the first proximal end 202 and the distal end 204) in a range of about 1 mm to about 1000 mm, about 2 mm to about 800 mm, about 3 mm to about 600 mm, about 4 mm to about 400 mm, about 5 mm to about 200 mm, about 10 mm to about 175 mm, about 15 mm to about 420 mm, about 20 mm to about 125 mm, about 25 mm to about 100 mm, about 30 mm to about 75 mm, or about 35 mm to about 50 mm. For example, the capillary 210 can include a length of at least about 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 110 mm, 120 mm, 130 mm, 140 mm, 420 mm, 160 mm, 170 mm, 180 mm, 190 mm, 200 mm, 300 mm, 400 mm, 500 mm, 600 mm, 700 mm, 800 mm, 900 mm, or 1000 mm.

In some embodiments, a side of the incomplete capillary 210 includes a thickness, width, or height in a range from about 0.05 mm to about 1.0 mm, from about 0.1 mm to about 1.0 mm, from about 0.5 mm to about 50 mm, from about 1 mm to about 45 mm, from about 1.5 mm to about 40 mm, from about 2 mm to about 35 mm, from about 2.5 mm to about 35 mm, from about 3 mm to about 30 mm, from about 3.5 mm to about 25 mm, from about 4 mm to about 20 mm, from about 4.5 mm to about 15 mm, or from about 5 mm to about 10 mm. For example, at least one of the sides 312, 314, and 316 can include a length of about any of: 0.05 mm, 0.1 mm, 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm.

In some embodiments, the thickness, width, or height of at least one side of the capillary 210 is the same through the entire length of the capillary (e.g. from a proximal end 202 to a distal end 204 of the capillary 210). In some embodiments, the capillary 210 includes at least two different thicknesses, widths, or heights through the entire length of the capillary (e.g. from a proximal end 202 to a distal end 204 of the capillary 210).

In some embodiments, two sides or planes of the incomplete capillary 210 includes a spacing (e.g., maximum spacing, minimum spacing, medium spacing, average spacing, or other spacing parameters) in a range from about 0.05 mm to about 1.0 mm, from about 0.1 mm to about 1.0 mm, from about 0.1 mm to about 50 mm, from about 1 mm to about 45 mm, from about 1.5 mm to about 40 mm, from about 2 mm to about 35 mm, from about 2.5 mm to about 35 mm, from about 3 mm to about 30 mm, from about 3.5 mm to about 25 mm, from about 4 mm to about 20 mm, from about 4.5 mm to about 15 mm, or from about 5 mm to about 10 mm. For example, the distance between the first portion 422 and the second portion 424 can include a length of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm. In some embodiments, the spacing is in a range of about 0.1 mm to about 0.5 mm, about 0.1 mm to about 0.4 mm, about 0.2 mm to about 0.4 mm, or about 0.2 mm to about 0.3 mm. In some embodiments, the spacing is about 0.1, 0.2, 0.3, 0.4, or 0.5 mm.

In some embodiments, the spacing is the same through the entire length of the capillary (e.g. from a proximal end 202 to a distal end 204 of the capillary 210). In some embodiments, the capillary 210 includes at least two different spacings through the entire length of the capillary.

In some embodiments, the incomplete capillary 210 includes at least one channel between two sides or planes for storing or transferring the collected samples.

In some embodiments, the channel includes a width in a range from about 0.05 mm to about 1.0 mm, from about 0.1 mm to about 1.0 mm, from about 0.05 mm to about 50 mm, from about 0.1 mm to about 45 mm, from about 1 mm to about 40 mm, from about 2 mm to about 35 mm, from about 2.5 mm to about 35 mm, from about 3 mm to about 30 mm, from about 3.5 mm to about 25 mm, from about 4 mm to about 20 mm, from about 4.5 mm to about 15 mm, or from about 5 mm to about 10 mm. For example, the distance between the first portion 422 and the second portion 424 can include a length of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm. In some embodiments, the channel includes a width in a range of about 0.1 mm to about 0.5 mm, about 0.1 mm to about 0.4 mm, about 0.2 mm to about 0.4 mm, or about 0.2 mm to about 0.3 mm. In some embodiments, the channel includes a width of about 0.1, 0.2, 0.3, 0.4, or 0.5 mm.

In some embodiments, the channel includes a depth in a range from about 0.05 mm to about 1.0 mm, from about 0.1 mm to about 1.0 mm, from about 0.05 mm to about 50 mm, from about 0.1 mm to about 45 mm, from about 1 mm to about 40 mm, from about 2 mm to about 35 mm, from about 2.5 mm to about 35 mm, from about 3 mm to about 30 mm, from about 3.5 mm to about 25 mm, from about 4 mm to about 20 mm, from about 4.5 mm to about 15 mm, or from about 5 mm to about 10 mm. For example, the distance between the first portion 422 and the second portion 424 can include a length of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm. In some embodiments, the channel includes a depth in a range of about 0.1 mm to about 0.5 mm, about 0.1 mm to about 0.4 mm, about 0.2 mm to about 0.4 mm, or about 0.2 mm to about 0.3 mm. In some embodiments, the channel includes a depth of about 0.1, 0.2, 0.3, 0.4, or 0.5 mm.

In some embodiments, the width of the channel is the same through the entire length of the capillary (e.g. from a proximal end 202 to a distal end 204 of the capillary 210). In some embodiments, the capillary 210 includes at least two different widths of the channel through the entire length of the capillary.

In some embodiments, the width of the channel is greater than the depth of the channel. In some embodiments, the width of the channel is greater than the depth of the channel throughout the channel. In some embodiments, the width and the depth of the channel are configured such that the channel includes a Urchin structure.

In some embodiments, the width of the channel is smaller than the depth of the channel. In some embodiments, the width of the channel is smaller than the depth of the channel throughout the channel.

In some embodiments, the capillary can hold a volume of samples in a range of about 0.1 ml to 20 ml, about 0.5 ml to 15 ml, about 1 ml to 10 ml, about 2 ml to 9 ml, about 3 ml to 8 ml, about 4 ml to 7 ml, or about 4 ml to 6 ml. In some embodiments, the channel can hold a volume of samples of at least about 0.1 ml, 0.2 ml, 0.3 ml, 0.4 ml, 0.5 ml, 0.6 ml, 0.7 ml, 0.8 ml, 0.9 ml, 1 ml, 1.5 ml, 2 ml, 2.5 ml, 3 ml, 3.5 ml, 4 ml, 4.5 ml, 5 ml, 5.5 ml, 6 ml, 6.5 ml, 7 ml, 7.5 ml, 8 ml, 8.5 ml, 9 ml, 9.5 ml, 10 ml, 11 ml, 12 ml, 13 ml, 14 ml, 15 ml, 16 ml, 17 ml, 18 ml, 19 ml, or 20 ml.

In some embodiments, the curved portion of the incomplete capillary 210 includes a diameter (e.g., maximum, minimum, medium, or average diameter) in a range from about 0.05 mm to about 1.0 mm, from about 0.1 mm to about 1.0 mm, from about 0.5 mm to about 50 mm, from about 1 mm to about 10 mm, or from about 1 mm to about 5 mm. For example, the curved shape 532 includes a diameter of about any of: 0.05 mm, 0.1 mm, 0.2 mm, 0.3 mm, 0.4 mm, 0.5 mm, 0.6 mm, 0.7 mm, 0.8 mm, 0.9 mm, 1 mm, 5 mm, 10 mm, or 50 mm.

In some embodiments, the diameter of the curved portion is the same through the entire length of the capillary (e.g. from a proximal end 202 to a distal end 204 of the capillary 210). In some embodiments, the capillary 210 includes at least two different diameters of the curved portion through the entire length of the capillary.

In various embodiments, the length, width, or height of at least one side or plane of the capillary tapers from a maximum at the proximal end 202 of the capillary 210 to a minimum at the distal end 204 of the capillary 210. In some embodiments, as shown in FIG. 2B, the distance between two sides 212 and 214 (or the width of the side 216) tapers from a maximum at the proximal end 202 of the capillary 210 to a minimum at the distal end 204 of the capillary 210.

In various embodiments, the length, width, or height of at least one side or plane of the capillary tapers from a maximum at the distal end 204 of the capillary 210 to a minimum at the proximal end 202 of the capillary 210.

In various embodiments, the maximum of such characteristics of the capillary 210 occurs at any point of the capillary 210 and taper to a minimum at the proximal end 202 and/or the distal end 204 of the capillary 210.

In various embodiments, the minimum of such characteristics occurs at any point of the capillary 210 and taper to a maximum at the proximal end 202 and/or the distal end 204 of the capillary 210.

In various embodiments, such characteristics of the capillary 210 alternates between a minimum and a maximum from the proximal end 202 to the distal end 204 of the capillary 210.

FIGS. 2A and 2B are images of various exemplary automation compatible collection devices 200, 300, 400, 500, and 600, according to various embodiments.

As shown in FIGS. 2A and 2B, in some embodiments, at least part of the collection device (e.g., 200, 300, 400, 500, or 600) includes an incomplete capillary 210, 310, 410, 510, or 610. In some embodiments, the incomplete capillary 210, 310, 410, 510, and/or 610 includes a cross section that can take any known form or structure with 1, 2, 3, or more sides.

For example, the incomplete capillary 210 includes a three-sided structure (e.g., a cross section including a double L form with three sides). In another example, the incomplete capillary 310 includes a parallel-plane structure (e.g., a cross section including a U-shape form including two parallel planes). In another example, the incomplete capillaries 410 and 510 include a curved (e.g., semi-circle or semi-ellipse) shape. polygonal (e.g., triangle) shape. In some embodiments, the cross-section of the incomplete capillary 210, 310, 410, 510, or 610 includes the same shape through the entire length of the capillary (e.g. from a proximal end to a distal end of the capillary). In some embodiments, the cross-section of the incomplete capillary 210, 310, 410, 510, or 610 includes at least two different shapes through the entire length of the capillary (e.g. from a proximal end to a distal end of the capillary).

In some embodiments, the capillary 210 or any of its components is compatible with standard wells for 1536-well plate formats, 384-well plate formats, 96-well plate formats, 48-well plate formats, 36-well plate formats, 24-well plate formats, 12-well plates formats, or any other standard well-plate formats.

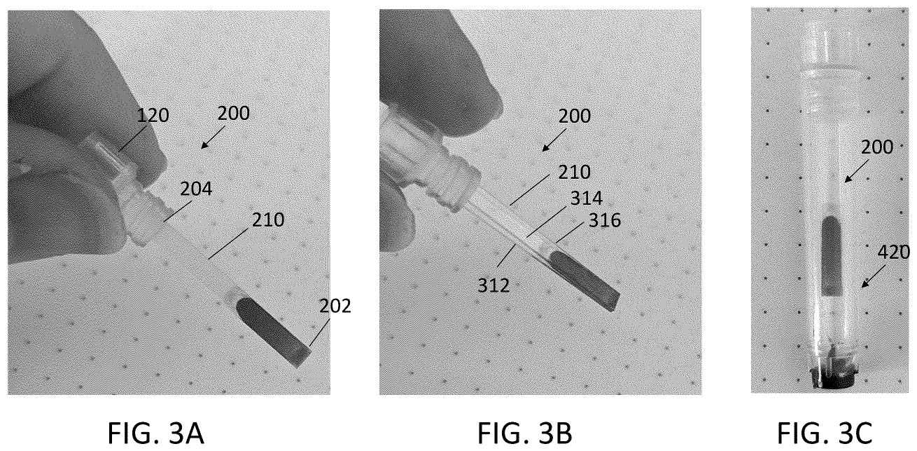

FIGS. 3A-3C are images of an exemplary automation compatible collection device 200. In some embodiments, the compatible collection device 200 includes an incomplete capillary 210 that is three-sided. For example, as shown in FIG. 3B, the capillary 210 includes a first side 312, a second side 314, and a third side 316.

In some embodiments, at least one of the sides 312, 314, and 316 of the incomplete capillary 210 includes a thickness, width, height, or spacing with respect to another side in a range of about 0.5 mm to about 50 mm, about 1 mm to about 45 mm, about 1.5 mm to about 40 mm, about 2 mm to about 35 mm, about 2.5 mm to about 35 mm, about 3 mm to about 30 mm, about 3.5 mm to about 25 mm, about 4 mm to about 20 mm, about 4.5 mm to about 15 mm, or about 5 mm to about 10 mm. For example, at least one of the sides 312, 314, and 316 can include a length of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm.

In some embodiments, the thickness, width, height, or spacing is the same through the entire length of the capillary (e.g. from a proximal end 202 to a distal end 204 of the capillary 210). In some embodiments, capillary includes at least two different thicknesses, widths, heights, or spacings through the entire length of the capillary.

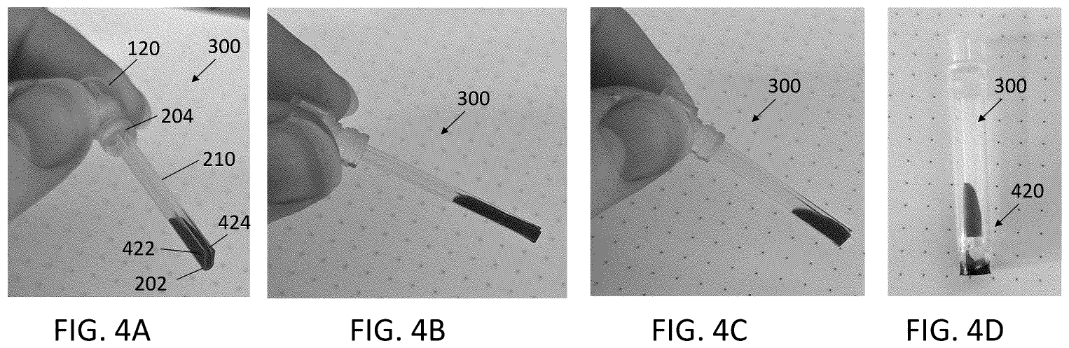

FIGS. 4A-4D are images of another exemplary automation compatible collection device 300. In some embodiments, the compatible collection device 300 includes an incomplete capillary 210 in a curved structure.

As shown in FIG. 4A, the capillary 210 includes a first portion 422 and a second portion 424. In some embodiments, the first portion 422 and a second portion 424 are parallel such that the incomplete capillary 210 is in a parallel form or structure. Accordingly, the first portion 422 and the second portion 424 are configured to have the same spacing throughout the height (e.g., the distance between the top and the bottom of the cross section) of the incomplete capillary 210. In some embodiments, the first portion 422 and a second portion 424 can be configured to form any nonparallel structure of the capillary 210. Accordingly, the first portion 422 and the second portion 424 are configured to have at least two different spacings throughout the height (e.g., the distance between the top and the bottom of the cross section) of the incomplete capillary 210.

In some embodiments, the spacing (e.g., maximum spacing, minimum spacing, medium spacing, or average spacing) between the first portion 422 and the second portion 424 is in a range of about 0.5 mm to about 50 mm, about 1 mm to about 45 mm, about 1.5 mm to about 40 mm, about 2 mm to about 35 mm, about 2.5 mm to about 35 mm, about 3 mm to about 30 mm, about 3.5 mm to about 25 mm, about 4 mm to about 20 mm, about 4.5 mm to about 15 mm, or about 5 mm to about 10 mm. For example, the distance between the first portion 422 and the second portion 424 can include a length of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm.

In some embodiments, the distance between the first portion 422 and the second portion 424 is the same through the entire length of the capillary (e.g. from a proximal end 202 to a distal end 204 of the capillary 210). In some embodiments, the distance between the first portion 422 and the second portion 424 tapers through the entire length of the capillary (e.g. from a proximal end 202 to a distal end 204 of the capillary 210. In some embodiments, the distance between the first portion 422 and the second portion 424 alternates through the entire length of the capillary (e.g. from a proximal end 202 to a distal end 204 of the capillary 210). In some embodiments, the maximum and/or minimum distance between the first portion 422 and the second portion 424 occurs at any point through the entire length of the capillary (e.g. from a proximal end 202 to a distal end 204 of the capillary 210).

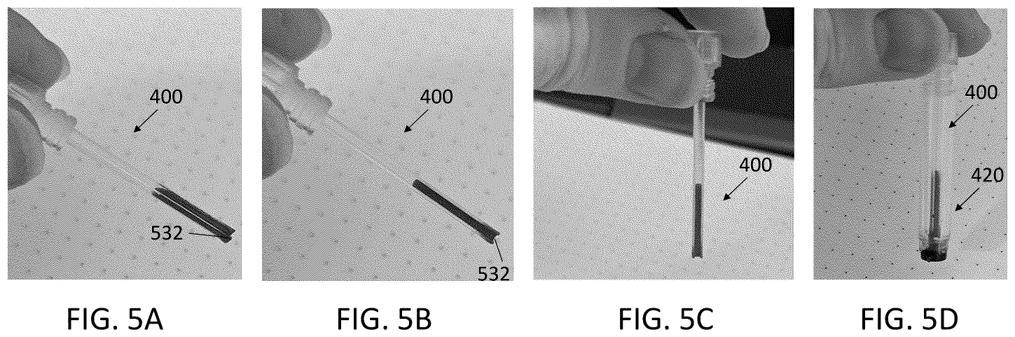

FIGS. 5A-5D are images of another exemplary automation compatible collection device 400. In some embodiments, the compatible collection device 400 includes an incomplete capillary 210 in a curved structure.

As shown in FIG. 5A-5C, the cross section of the incomplete capillary 210 includes a curved shape 532 (e.g., semi-circle).

In some embodiments, the curved shape 532 includes a diameter (e.g., maximum, minimum, medium, or average diameter) in a range of about 0.5 mm to about 50 mm, about 1 mm to about 45 mm, about 1.5 mm to about 40 mm, about 2 mm to about 35 mm, about 2.5 mm to about 35 mm, about 3 mm to about 30 mm, about 3.5 mm to about 25 mm, about 4 mm to about 20 mm, about 4.5 mm to about 15 mm, or about 5 mm to about 10 mm. For example, the curved shape 532 includes a diameter of about any of: 0.5 mm, 1 mm, 1.5 mm, 2 mm, 2.5 mm, 3 mm, 3.5 mm, 4 mm, 4.5 mm, 5 mm, 5.5 mm, 6 mm, 6.5 mm, 7 mm, 7.5 mm, 8 mm, 8.5 mm, 9 mm, 9.5 mm, 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, or 50 mm.

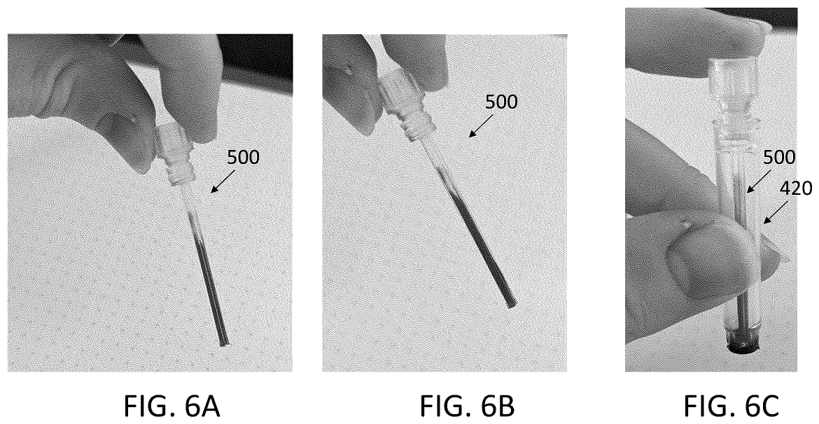

FIGS. 6A-6C are images of another exemplary automation compatible collection devices. As compared with FIGS. 6A-6C, the length of the capillary 210 is longer, and the curved portion 532 includes a smaller diameter.

In some embodiments, the performance of the complete capillary (e.g., capillary 110) can be improved by adapting the length of the capillary, dimensions and/or structures of the capillary.

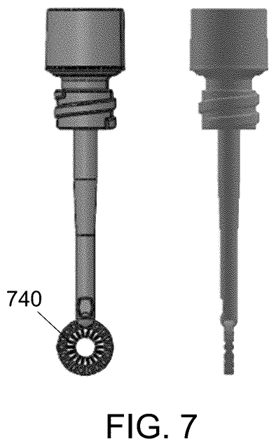

FIGS. 7-14 are schematic views of various exemplary automation compatible collection devices, according to various embodiments. In particular embodiments, the automation compatible collection device, as shown in FIGS. 7-14, includes an automation compatible cap (e.g., cap 120) and a capillary (e.g., capillary 110 or 210) including various structures, as described herein.

Referring to FIG. 7, in some embodiments, the incomplete capillary includes one or more cavities (e.g., cavities 740). In some embodiments, the cavities are configured to form a loop at or near the proximal end of the capillary (e.g., the end away from the cap). In some embodiments, the cavities are open to at least one side of the incomplete capillary. In some embodiments, the cavities include features to capture blood inside at least one cavity. In some embodiments, such features can allow fast drying of blood for transport, and/or other improved performance as described herein.

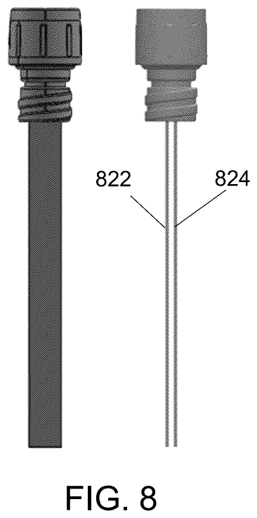

Referring to FIGS. 8 and 9, in some embodiments, the incomplete capillary includes parallel plates (e.g., plates 822 and 824 in FIGS. 8 and 9) to wick (or absorb) sample into the channel (e.g., channel 826) between the plates. Such parallel plates results in a three-sided U-shape cross-section (e.g., from a bottom view 940) of the capillary). In some embodiments, the capillary in FIGS. 8 and 9 includes the same structure as the capillary as discussed in FIGS. 4A-4D. Accordingly, a volume of samples collected can be determined by the width of each plate and the distance between the two plates.

Referring to FIG. 10, in some embodiments, the incomplete capillary includes a cross-section of V-shape (e.g., V-shape cross-section 1040). Such cross-section results in a V-shaped channel that can wick (or absorb) sample into the sample collection device. Accordingly, a volume of samples collected can be determined by the width or depth of the channel.

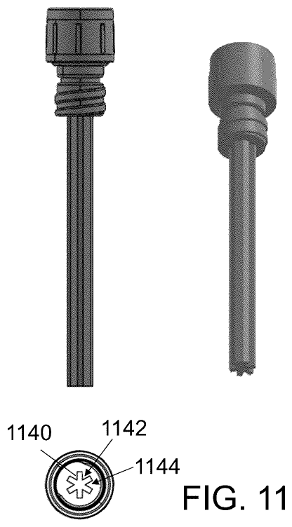

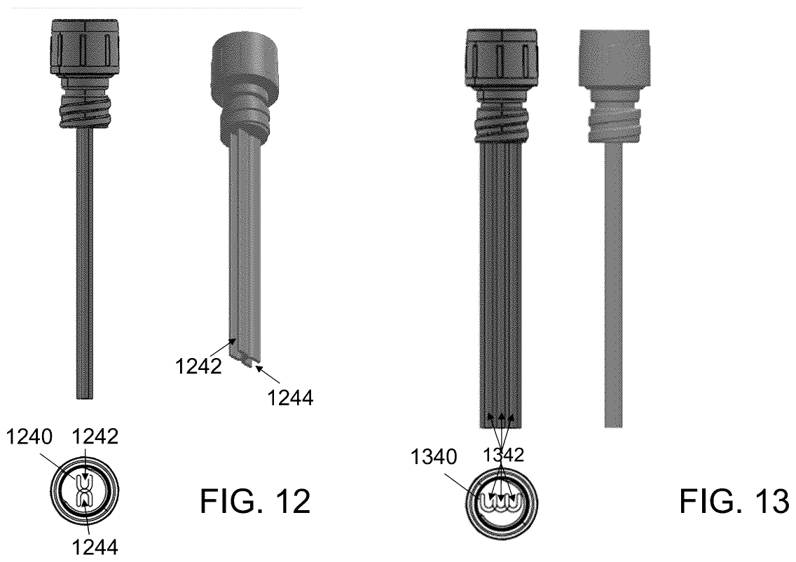

In some embodiments, as shown in FIGS. 11-13, the incomplete capillary includes a plurality of plates, a plurality of sides, and/or a cross-section including various shapes, resulting in multiple cavities on the outside surface of the capillary. Accordingly, a volume of samples collected can be determined by the width or depth of the multiple cavities. For example, as shown in FIG. 11, the cross-section of the incomplete capillary includes a plurality of V-shapes (e.g., multi-V shaped cross-section 1140 in FIG. 11. Such configuration can wick (or absorb) sample into the cavities (e.g., cavities 1142 and 1144) along the outside surface of the device e.g., via capillary action. In another example, as shown in FIG. 12, the cross-section (e.g., cross-section 1240) of the incomplete capillary includes two U-shapes. Such configuration can wick (or absorb) sample into the cavities (e.g., cavities 1242 and 1244) along the outside surface of the device e.g., via capillary action. In another example, as shown in FIG. 13, the cross-section (e.g., cross-section 1340) of the incomplete capillary includes three U-shapes. Such configuration can wick (or absorb) sample into the cavities (e.g., cavities 1342) along the outside surface of the device e.g., via capillary action.

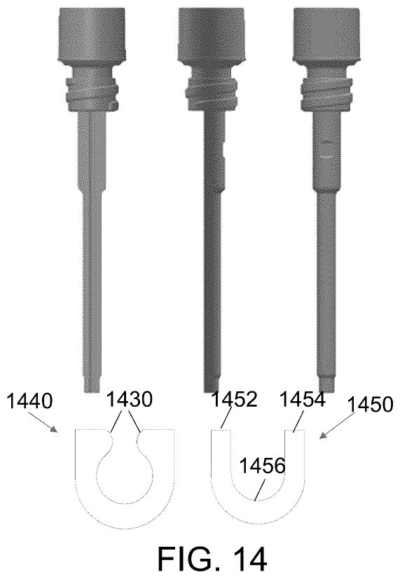

In some embodiments, as shown in FIG. 14, the incomplete capillary (e.g., capillary 1440) is configured to include round overhangs e.g., 1430. In comparison with the capillary that has a cross-section 1450 and three sides 1452, 1454, and 1456, the configuration of round overhangs 1430 can be beneficial for transport.

FIG. 35 is an image of an exemplary automation compatible collection device including an incomplete capillary structure. As shown in FIG. 35, the incomplete capillary demonstrates a successful performance in collecting blood through substantially the entire length of the incomplete capillary structure.

Cap

In some embodiments, the collection device further includes a cap (e.g., cap 120 in FIGS. 1-6). In some embodiments, the cap is configured to be attached to one end (e.g., the distal end) of the capillary (e.g., capillary in FIGS. 1-14). In some embodiments, the cap is operatively, removably, and/or firmly coupled (e.g., attached) to the capillary. In some embodiments, the cap is irremovable and locked with the capillary. In some embodiments, the cap or a portion of the cap can be formed as a single molded part (e.g., a unitary part or item) and/or as separate parts. In some embodiments, the cap can be used as a handle by the person using the collection device (e.g., before the collection device is broken and shortened). In some embodiments, the cap is adapted to fit any standard or custom vial and/or compatible to any automation process.

In some embodiments, the cap includes interior or exterior features (e.g., ring or bubble blower, urchin structures) configured to collect and/or hold fluids.

In some embodiments, the cap is adapted to any collection device (e.g., capillary as described herein, swab, lancet, or other suitable devices).

In some embodiments, the cap comprises a structure and/or configuration adapted to interface with an automation device (e.g., a tube capper, a handheld or fully automated decapper machine). In general, the cap can have any structure that corresponds with any known or future developed automation device (e.g., manufactured by Abbott™ ThermoFisher™, etc.). For example, in some embodiments, the cap comprises a hollow internal portion, e.g., that interfaces with an automated device. In some embodiments, an outer surface of the cap (e.g., top surface, circumferential surface, side surface) interfaces with an automation device. In some embodiments, the proximal end of the cap (i.e., farther away from the proximal end) defines an opening leading to hollow internal portion of the cap. In some embodiments, the cap comprises a hollow cylinder. In some embodiments, the cap is defined by an outer cross-section (i.e., the external shape of the cap) and an inner cross-section (i.e., the internal shape of the hollow portion). In some embodiments, the outer and/or inner cross-section of the cap is a circle, a semicircle, a truncated circle, or a circle with one or more flat sides. In some embodiments, the outer and/or inner cross-section of the cap is a circle. In some embodiments, the outer and/or inner cross-section of the cap comprises a polygonal cross section, e.g., a cross-section in the shape of a triangle, a square, a quadrilateral, a trapezoid, a pentagon, a hexagon, a star, or a polygon with at least 7, 8, 9, 10, 11, 12, 13, 14, 15, 16, 17, 18, 19, 20 or more sides. In some embodiments, at least one side of the outer and/or inner cross-section of the cap comprises a convex and/or concave curve. In some embodiments, the outer and/or inner cross section of the cap is a rotationally symmetric shape. In some embodiments, the outer and/or inner cross section of the cap is an asymmetric shape. In some embodiments, the outer and/or inner cap cross-section is the same for the entirety of the cap. In some embodiments, the outer and/or inner cap cross-section is different for at least one portion of the cap; the cap can comprise any combination of different (e.g., at least 2, at least 3, at least 4, at least 5) cap cross-sections. In some embodiments, the outer and inner cap cross-sections of the cap are the same. In some embodiments, the outer and inner cap cross-sections of the cap are different.

In some embodiments, the cap comprises at least one (e.g., 1, 2, 3, 4, 5, or more) internal groove(s). In some embodiments, the cap comprises at least one (e.g., 1, 2, 3, 4, 5, or more) internal ridge(s). In some embodiments, the cap comprises at least one (e.g., 1, 2, 3, 4, 5, or more) external groove(s). In some embodiments, the cap comprises at least one (e.g., 1, 2, 3, 4, 5, or more) external ridge(s). In some embodiments, the internal or external groove(s) or internal ridge(s) are parallel with the axial shaft of the collection device.

In some embodiments, the cap can interface with an automated device. In some embodiments, the automated device can move, control, manipulate, etc. the collection device after interfacing with the cap. In some embodiments, a portion of an automated device can extend into the hollow internal portion of the cap. In some embodiments, hollow portion and the internal groove(s) or internal ridge(s) permit the cap to interface with an automated device. In some embodiments, the automated device is a tube capper and decapper machine. In some embodiments, the cap can be adjusted to fit any standard or custom tube that is compatible with the SBS 24-well format, the SBS 48-well format, the SBS 96-well format, or any combination thereof. In some embodiments, the cap can be adjusted for any automation format. In some embodiments, formats used and/or adjusted for may include a large format and/or a universal format with 12, 24, 36, 48 and/or other rack sizes.

In some embodiments, the capillary as described herein does not interfere with interaction between the cap and the automation device. For example, upon in use, the hollow interior of cap would be unimpeded by the capillary.

In some embodiments, the cap is removably, operatively, and/or firmly coupled (or connected, attached) to the capillary or other collection devices and can be freely detached from the capillary.

In some embodiments, the cap 120 is translatable along the capillary or other collection devices. For example, the cap 120 can move from one position to another position between the proximal end and distal end of the capillary. In some embodiments, the cap 120 is configured to stay attached to (e.g., locked at) the capillary (e.g., before or after the sample is collected). In some embodiments, after the collection device is inserted into a vial, the cap 120 stays attached to the capillary in the vial (e.g., a vial 420 in FIGS. 1 and 3-6). In some embodiments, during an automation process (e.g., decapping), the cap 120 is configured to be detached from the capillary 100, in response to an application of an external force from e.g., an automated device as described herein.

In some embodiments, the cap includes a threaded portion 122 (e.g., a cylindrical portion including one or more raised helical threads). In some embodiments, the cap is operatively coupled to the threaded portion 122 (e.g., a cylindrical portion including one or more raised helical threads). In some embodiments, the threaded portion 122 is configured to interface with a threaded portion of a vial (or container tube) for sealing the cap 120 with the vial.

In some embodiments, the threaded portion 122 is hollow, and the proximal end of the threaded portion 122 defines an opening leading to an hollow internal portion in the threaded portion 122 of the cap 120. Thus, the threaded portion 122 of the cap 120 can receive and operatively coupled to the capillary.

In some embodiments, the threaded portion 122 includes at least 1, 2, 3, 4, or 5 threads 123. In some embodiments, the threads 123 are external threads located on the outside of the threaded portion 122. In other embodiments, the threads 123 are internal threads located inside the threaded portion 122. In some embodiments, the threads 123 are continuous, discontinuous, or a combinations thereof.

In some embodiments, the threaded portion 122 includes a geometry, pitch, direction, number, and/or dimensions of the threads that matches the threaded portion of the vial such that the cap 120 can be fully, firmly and/or securely screw in place to the vial. In some embodiments, the vial includes a width or diameter in a range from about 1 mm to about 20 mm, from about 5 mm to about 15 mm, from about 5 mm to about 15 mm, from about 12 mm to about 14 mm, or from about 6 mm to about 10 mm. In some embodiments, the vial includes a height or length in a range from about 10 mm to about 200 mm, from about 40 mm to about 100 mm, from about 40 mm to about 60 mm, from about 60 mm to 100 mm, or from about 65 mm to about 100 mm.

In some embodiments, the threaded portion 122 includes an inner diameter (e.g., a diameter of the opening) and/or an outer diameter, which are substantially equal to or greater than the average diameter of the capillary. In some embodiments, the inner diameter and/or the outer diameter of the threaded portion 122 is from about 0.5 mm to about 10 mm.

In some embodiments, the cap 120 includes an annular portion 124 (e.g., a cylindrical portion) attached to the threaded portion 122. When the cap is attached to the capillary, the threaded portion 122 is closer to the incomplete capillary than the annular portion 124.

In some embodiments, the annular portion 124 is hollow, and the proximal end of the annular portion 124 defines an opening 128 leading to an hollow internal portion of the annular portion 124 of the cap 120. Thus, the annular portion 124 of the cap 120 can receive and operatively coupled to the capillary. In some embodiments, the annular portion 124 includes an inner diameter (e.g., a diameter of the opening 128) and/or an outer diameter. In some embodiments, the inner diameter and/or outer diameter of the annular portion 124 is greater than the diameter of the capillary, the diameter of the opening of the cap 120, and/or the outer parameter of the threaded portion 122. In some embodiments, the inner diameter and/or the outer diameter of the annular portion 124 is from about 0.05 mm to about 10 mm. In some embodiments, the outer diameter of the annular portion 124 defines the maximum diameter of the cap 120. In some embodiments, the annular portion 124 includes a solid interior (e.g., not hollow).

In some embodiments, the cap may be locked and stay attached to the capillary without having the mobility (e.g., along any direction). Upon being acted upon by an external force (e.g., from a decapper machine or other suitable automation devices) the cap can be removed from the vial. In some embodiments, the cap is configured to remain attached to the capillary upon being acted upon by an automation device (e.g., such that upon removal of the cap from the vial, so is the capillary). In such instances, a testing process can occur using either the cap attached to the capillary (e.g., by transporting the cap/capillary into an assay solution) and/or a testing process can occur using the material left behind in the vial (e.g., with the cap and capillary removed from the vial, the assay solution can be delivered into the vial). In other embodiments, the automation device can detach the cap from the capillary, such that the capillary remains behind in the vial while the assay solution is delivered. In either embodiment, following the application of an assay solution (or other testing protocol), the cap can either be resecured to the vial for storage/incubation of the tests or the cap can be disposed of and a different cap can be used for storage/incubation.

In some embodiments, the cap is configured to interface with an automation device (e.g., a decapper machine). For example, the cap includes at least one (e.g., 1, 2, 3, 4, 5, or more) internal ribs to enable an operation by an automation device. In some embodiments, the internal ribs are parallel with the axial shaft of the capillary. In some embodiments, the internal ribs includes a height less than or equal to the height of the annular portion of the cap. In some embodiments, the internal ribs includes a diameter less than the inner diameter of the annular portion of the cap.

In some embodiments, the cap is configured to be attached to the capillary firmly or securely in place, by applying an anti-rotation feature (not shown). In some embodiments, the anti-rotation feature is located at the inner wall of the annular portion, or any suitable position within the cap.

In some embodiments, the cap can be adjusted to fit any standard or custom vial that is compatible with the SBS 24-well format, the SBS 48-well format, the SBS 96-well format, or any combination thereof. In some embodiments, the cap can be adjusted for any automation format, including large format/universal sized tube racks for 12, 24, 36, and 48 tube arrays

In some embodiments the caps is supplied separate from the capillary. In some embodiments, the cap is supplied with the capillary.

Additional Features or Elements

In some embodiments, the collection device includes one or more additional features or elements, such as one or more textured channels, sub-channels, indicators, capillary tips, and cap capillary assembly features, as described herein. As used herein, the term “channel” refers to a portion of a capillary where the fluids are collected, stored, conveyed through, or transported. In some embodiments, the term “channel” refers to the “cavities” as shown in e.g., FIGS. 8-11.

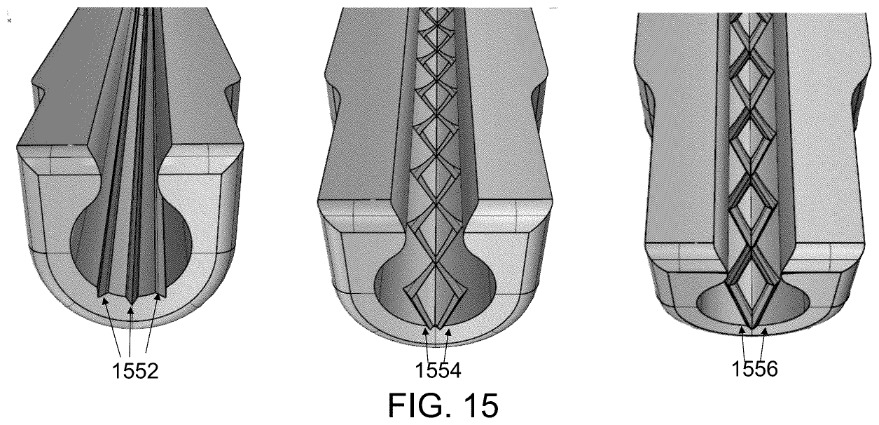

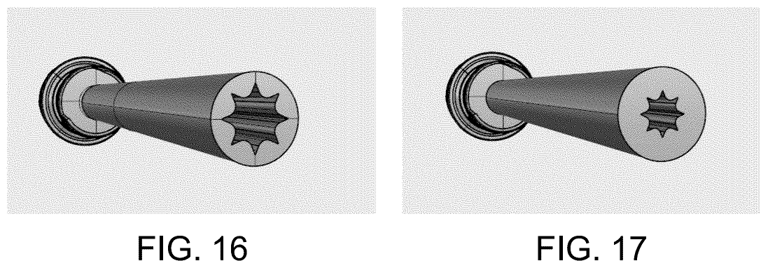

In some embodiments, the capillary (e.g., complete capillary or incomplete capillary) of the collection device includes one or more channels (or “cavities” as shown in e.g., FIGS. 8-11), where the channel includes textures to improve sample travelling through the channel. For example, as shown in FIG. 15, the channel texturing include one or more sub-channels (or secondary channels) (e.g. sub-channels 1552, 1554, or 1556). In some embodiments, the sub-channel can include any structures or configurations. In another example, as shown in FIGS. 16 and 17, the capillary can include a textured channel with uneven surface in the interior.

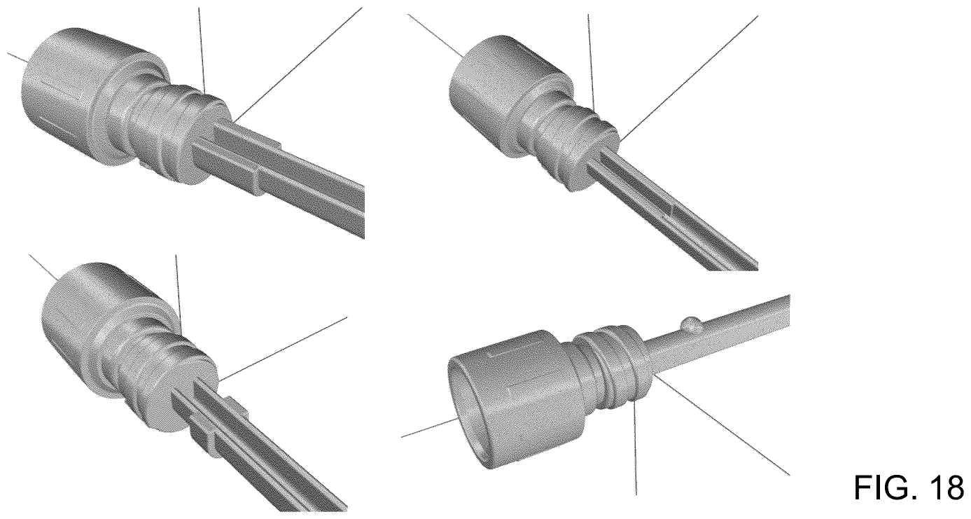

Referring to FIG. 18, in some embodiments, the collection device includes one or more indicators (e.g., volume indicators) at or near the distal end (the end closer to the cap) of the capillary. The indicators can provide a visual signal indicating that the sample collected in the capillary reaches the indicator line. Thus, the indicators can alert a user when a target volume of collected fluid is achieved. In some embodiments, the indicator can be an element attached to the outside surface of the capillary. In some embodiments, the indicator can be an element attached to the inside surface of the capillary. In some embodiments, the indicator extends to the cap. In some embodiments, the indicator can be a protuberance at the outside surface of the capillary. In some embodiments, the indicator can include any configurations.

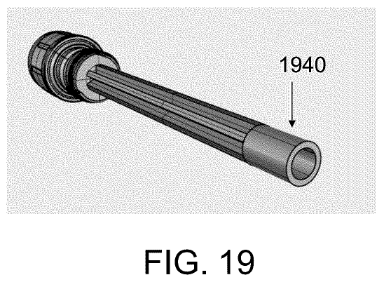

Referring to FIG. 19, in some embodiments, the collection device further includes a capillary tip (e.g., capillary tip 1940) operatively coupled to the proximal end of the capillary. In some embodiments, the capillary tip transfers sample into one or more channels, while it provides increased patient comfort and safety.

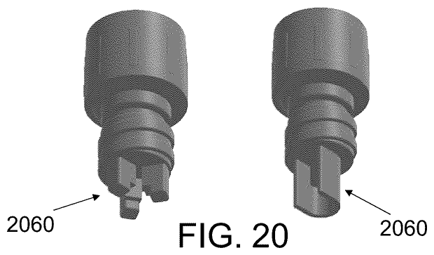

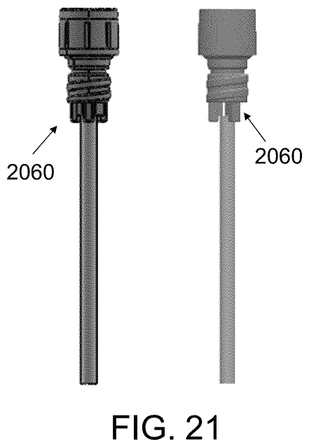

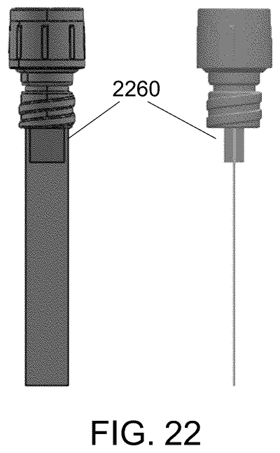

Referring to FIGS. 20-22, in some embodiments, the collection device further includes an assembly feature (e.g., element 2060 or 2260) to accommodate assembly of sample collection medium (e.g., an FTA® paper) for providing a final collection device. The assembly feature couples the capillary to the cap operatively and firmly to provide a final collection device using an assembly method e.g., press-fit, glue, ultrasonic welding, rivet, or any other methods.

In some embodiments, the assembly feature includes one or more pins, a flat plate, a secondary cap of any shapes, or any other configurations. In some embodiments, a sample collection medium, such as an FTA® paper, is assembled into the cap for collection of biological samples.

In some embodiments, the collection device includes a component or surface treatment that separates the different components of blood (e.g., cellular, bacterial, or viral nucleic acids, proteins, antibodies, or polypeptides).

In some embodiments, the collection device includes a series of interior or exterior features (e.g., a ring or bubble blower, and/or urchin structures) configured to collect and/or hold fluids.

Referring to FIGS. 23 and 24, in some embodiments, the collection device in the present disclosure includes one or more mechanical devices, such as a suction device, a duck-bill valve, and/or other suitable devices for sample collection and/or retention of samples.

In some embodiments, the suction device can be operatively coupled to either or both ends of the cap to aid in collecting samples (e.g., blood or other fluids) by suctioning. For example, as shown in FIG. 23, the suction device 2360 is operatively attached to a first end (e.g., opening 128) of the cap 120, where the first end is away from the proximal end 2302 of the sample collection vehicle 2310. In another example, as shown in FIG. 24, the suction device 2360 is operatively attached to a second end of the cap 120, where the second end is closer to the proximal end 2302 of the sample collection vehicle 2310.

In some embodiments, the suction device can be operated by hand or by a machine (e.g., automated machine).

In some embodiments, the suction device is removable from the cap to keep the automation compatibility of the collection device. In some embodiments, the suction device is locked with the cap in use, and can be detached after samples are collected.

Optionally, the suction device includes at least one frangible connection (or breakpoint) for removing the suction device by applying an external force. In some embodiments, the frangible connection has a lower strength under shear, torsional, and/or compressive forces than the other portions of the suction device. In some embodiments, an external force includes a single direction bend, torsion, twisting, or any combinations thereof, at the same time or at different times. For example, a user could snap off, twist off, pull off, or otherwise cut the suction device at the frangible connection.

As shown in FIG. 24, the collection device can further include a duck-bill valve 2460 to aid in sample collection and/or retention of samples during transport. In some embodiments, the duck-bill valve 2460 is attached to the proximal end 2302 of the sample collection vehicle 2310. In some embodiments, the duck-bill valve 2460 is located in proximity to the proximal end 2302 of the sample collection vehicle 2310.

In some embodiments, the sample collection vehicle 2310 as shown in FIGS. 23 and 24 refer to a capillary or other sample collection structures as described herein.

Stem and Collection Head

In various embodiments, the capillary structure described in above embodiments can be replaced by a stem and/or a sample collection head for collecting biological samples.

Accordingly, a collection device can include a sample collection head, a stem, a cap, and/or any other elements, as described herein. While further exemplary description of such embodiments will be described below, this application expressly contemplates any of the embodiments described above with the capillary structure being replaced by any sample collection head described below.

In general, the sample collection head, as described herein can include any structures, such as fins (e.g., parallel fins), flat surfaces (e.g., parallel and/or interconnecting flat surfaces), nubs (e.g., nubs including hemisphere surfaces, or nubs including pyramid structure), bars (e.g., parallel bars), combinations thereof, and/or any other porous or non-porous structures to collect biological samples.

Advantageously, the structures of the sample collection head as described herein can result in improved performance in collecting biological samples (e.g., cells) by generating larger friction and/or larger surface area compared with traditional collection devices. For example, the sample collection head as described herein can collect (e.g., obtain, retain, hold, and/or store) larger amounts of DNA than a traditional collection device (e.g., a regular swab). In some embodiments, the sample collection head includes one or more smooth and/or regular surfaces to achieve an improved performance in collecting biological samples. In some embodiments, the sample collection head includes one or more rough, uneven, and/or irregular surfaces an improved performance in collecting biological samples. In some embodiments the sample collection head incorporates a brush-head structure. In some embodiments, the biological samples are collected from a surface area of a mouth, or any other suitable area.

In some embodiments, the stem and/or the sample collection head include one or more features or elements, such as a self-locking feature, an anti-rotation feature, and/or any other features a self-locking feature, an anti-rotation feature, and/or any other features, so as to firmly and securely coupled to each other or another portion (e.g., the cap) of the collection device.

As used herein, the term “locked,” “locking,” or “anti-rotation” refers to any condition in which there is greater resistance to movement of the cap along the stem than at non-locked positions.

FIGS. 26-33 are schematic, detail views of various exemplary sample collection heads of automation compatible collection devices for collecting biological samples, according to various embodiments, as described herein. In some embodiments, the biological samples include DNA, RNA, protein, cellular, wound, pox, tissue, and/or any other biological samples. In general, a stem includes an axial shaft (e.g., a solid cylindrical rod). In some embodiments, the stem 2610 includes a solid interior. In some embodiments, the stem 2610 includes an hollow interior. In some embodiments, the stem 2610 is mechanically and/or firmly coupled (e.g., attached) to the cap 120. In some embodiments, the stem 2610 is removably coupled to a cap 120. In some embodiments, the cap 120 is not removable and is locked with the stem 2610. In some embodiments, the cap 120 is operatively coupled to any portion of the collection device (e.g., sample collection head). In some embodiments, the sample collection head 2620 is applicable to any collection device (e.g., blood collection device, biological sample collection device, swabs, and/or other sample collection devices from any part of a biological location of a subject). In some embodiments, the stem 2610 can be replaced by a capillary as described above, or by any other suitable structures. In some embodiments, the stem 2610 is not included in an automation compatible collection device. For example, the sample collection head can be directly coupled to the cap 120 without a stem.

In some embodiments, the stem 2610 can include a cross-section of at least one shape of a polygonal shape, e.g., a triangle, a square, a quadrilateral, a trapezoid, a pentagon, a hexagon, a polygon with at least 7, 8, 9, 10, 11, 12, 13, 14, 15, 16, 17, 18, 19, 20 or more sides, a circle, a circle with one or more flat sides, an ellipse, or an ellipse with one or more flat sides. In some embodiments, at least one side of the cross-section includes a convex and/or concave curve. In some embodiments, the cross-section includes a rotationally symmetric shape or an asymmetric shape. In some embodiments, the cross-section includes the same shape, or at least two different shapes for the entirety of the component.

In some embodiments, the stem 2610 includes a proximal end 2612 and a distal end 2614. In some embodiments, the proximal end 2612 is coupled to the sample collection head 2620, and the distal end 2614 is coupled to the cap 120. In some embodiments, the proximal end 2612 and/or the distal end 2614 include any suitable shape, e.g., rounded, pointed, grooved, and/or other suitable shapes.

In some embodiments, the stem 2610 includes an open end at one or both ends (e.g., at the proximal end 2612 and/or the distal end 2614) of the stem 2610. In some embodiments, the stem 2610 is configured to include a closed end at one or both ends (e.g., at the proximal end 2612 and/or the distal end 2614) of the stem 2610.

In some embodiments, the stem 2610 includes a length (e.g., distance between the proximal end 2612 and the distal end 2614) in a range from about 1 mm to about 200 mm, from about 1 mm to about 5 mm, from about 5 mm to about 200 mm, from about 10 mm to about 175 mm, from about 15 mm to about 420 mm, from about 20 mm to about 125 mm, from about 25 mm to about 100 mm, from about 30 mm to about 75 mm, or from about 35 mm to about 50 mm. For example, the stem 2610 includes a length of about: 1 mm, 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 110 mm, 120 mm, 130 mm, 140 mm, 420 mm, 160 mm, 170 mm, 180 mm, 190 mm, or 200 mm.

In some embodiments, the stem 2610 includes an outer diameter in a range from about 0.1 mm to about 6 mm, about 0.5 mm to about 5.5 mm, about 1.0 mm to about 5 mm, about 1.5 mm to about 4.5 mm, about 2 mm to about 4 mm, about 2.5 mm to about 3.5 mm, about 1 mm to about 50 mm, about 5 mm to about 45 mm, about 10 mm to about 40 mm, about 15 mm to about 35 mm, about 20 mm to about 30 mm. In some embodiments, the stem 2610 includes an outer diameter of about any of: 0.1 mm, 0.5 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, 15 mm, 20 mm, 25 mm, 30 mm, 35 mm, 40 mm, 45 mm, or 50 mm. In some embodiments, the stem 2610 includes an outer diameter of less than about any of: 0.1 mm, 0.5 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, 15 mm, 20 mm, 25 mm, 30 mm, 35 mm, 40 mm, 45 mm, or 50 mm.

In some embodiments, the stem 2610 includes substantially the same outer diameter for the entirety of the stem 2610. In various embodiments, the stem 2610 includes at least two different outer diameters for the entirety of the stem 2610. For example, the diameter of a first portion (e.g., the proximal end 2612) of the stem 2610 may be different from the diameter of a second portion (e.g., the distal end 2614) of the stem 2610.

In some embodiments, the stem 2610 is hollow and includes substantially the same inner diameter for the entirety of the stem 2610. In various embodiments, the stem 2610 includes at least two different inner diameters for the entirety of the stem 2610. For example, the diameter of a first portion of the hollow interior (e.g. at the proximal end 2612) of the stem 2610 may be different from the diameter of a second portion of the hollow interior (e.g., at the distal end 2614) of the stem 2610.

In some embodiments, the stem 2610, or at least a portion of the stem 2610 tapers (e.g., has sequentially reduced outer diameters and/or inner diameters) towards the proximal end 2612, and/or the distal end 2614, from the middle of the stem 2610 or any point of the stem 2610.

In some embodiments, the stem 2610 tapers from a maximum diameter at the proximal end 2612 of the stem 2610 to a minimum diameter at the distal end 2614 of the stem 2610.

In some embodiments, the stem 2610 tapers from a maximum diameter at the distal end 2614 of the stem 2610 to a minimum diameter at the proximal end 2612 of the stem 2610.

In some embodiments, the maximum diameter of the stem 2610 occurs at any point of the stem 2610 and the diameters taper to a minimum diameter at the proximal end 2612 and/or the distal end 2614 of the stem 2610.

In some embodiments, the minimum diameter of the stem 2610 occurs at any point of the stem 2610 and the diameters taper to a maximum diameter at the proximal end 2612 and/or the distal end 2614 of the stem 2610.

In some embodiments, the diameter of the stem 2610 alternates between a minimum diameter and a maximum diameter from the proximal end 2612 to the distal end 2614 of the stem 2610.

In some embodiments, the performance of the stem 2610 can be improved by adapting dimensions or sizes (length, outer diameter), shapes and/or structures of the stem 2610.

In general, the sample collection head can be operatively and/or firmly coupled (e.g., attached) to the stem. In some embodiments, the sample collection head is removably coupled to the stem. In some embodiments, the sample collection head is not removable and locked with the stem. In some embodiments, the sample collection head can be adapted to be operatively coupled to any portion of the collection device (e.g., the cap). In some embodiments, the sample collection head is applicable to any collection device (e.g., blood collection device, biological sample collection device, swabs, and/or other sample collection devices from any part of a biological location of a subject). In some embodiments, the sample collection head is replaced by a capillary as described above, a swab, or any other sample collection structure.

In some embodiments, the sample collection head includes a cross-section of at least one shape of a polygonal shape, e.g., a triangle, a square, a quadrilateral, a trapezoid, a pentagon, a hexagon, a polygon with at least 7, 8, 9, 10, 11, 12, 13, 14, 15, 16, 17, 18, 19, 20 or more sides, a circle, a circle with one or more flat sides, an ellipse, or an ellipse with one or more flat sides. In some embodiments, at least one side of the cross-section includes a convex and/or concave curve. In some embodiments, the cross-section includes a rotationally symmetric shape or an asymmetric shape. In some embodiments, the cross-section includes the same shape, or at least two different shapes for the entirety of the component. In some embodiments, the cross section includes cavities for collecting and retaining sample.

In some embodiments, the performance of the sample collection head can be improved by adapting dimensions or sizes (length, outer diameter), shapes and/or structures of the sample collection head.

Referring still to FIGS. 26-33, in some embodiments, the sample collection head 2620 includes a proximal end 2622 and a distal end 2624. In some embodiments, the proximal end 2622 is used to collect sample from a sample collection area or biological material, and the distal end 2624 is coupled to the stem 2610. In some embodiments, the proximal end 2622 and/or the distal end 2624 include any suitable shape, e.g., rounded, pointed, grooved, carved, and/or other suitable shapes.

In some embodiments, the sample collection head 2620 includes an open end at one or both ends (e.g., at the proximal end 2622 and/or the distal end 2624) of the sample collection head 2620. In some embodiments, the sample collection head 2620 includes a closed end at one or both ends (e.g., at the proximal end 2622 and/or the distal end 2624) of the sample collection head 2620.

In some embodiments, the sample collection head 2620 includes a length (e.g., distance between the proximal end 2622 and the distal end 2624) in a range of about 5 mm to about 200 mm, about 10 mm to about 175 mm, about 15 mm to about 420 mm, about 20 mm to about 125 mm, about 25 mm to about 100 mm, about 30 mm to about 75 mm, or about 35 mm to about 50 mm. For example, the stem 2610 can include a length of about any of: 5 mm, 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, 110 mm, 120 mm, 130 mm, 140 mm, 420 mm, 160 mm, 170 mm, 180 mm, 190 mm, or 200 mm. In particular embodiments, as shown in FIG. 33, the sample collection head 2620 includes a length of about 22.5 mm or 30 mm.

In some embodiments, the sample collection head 2620 (or a portion thereof) includes a size (e.g., width, height, outer diameter, inner diameter, or other types of sizes) in a range from about 0.1 mm to about 6 mm, about 0.5 mm to about 5.5 mm, about 1.0 mm to about 5 mm, about 1.5 mm to about 4.5 mm, about 2 mm to about 4 mm, about 2.5 mm to about 3.5 mm, about 1 mm to about 50 mm, about 5 mm to about 45 mm, about 10 mm to about 40 mm, about 15 mm to about 35 mm, about 20 mm to about 30 mm. In some embodiments, the sample collection head 2620 (or a portion thereof) includes a size (e.g., width, height, outer diameter, inner diameter, or other types of sizes) of about: 0.1 mm, 0.5 mm, 1 mm, 1.5 mm, 2 mm, 2.5 mm, 3 mm, 3.5 mm, 4 mm, 4.5 mm, 5 mm, 6 mm, 7 mm, 8 mm, 9 mm, 10 mm, 15 mm, 20 mm, 25 mm, 30 mm, 35 mm, 40 mm, 45 mm, or 50 mm.

In some embodiments, the sample collection head 2620 (or a portion thereof) includes the same width for the entirety of the sample collection head 2620. In various embodiments, the sample collection head 2620 includes at least two different widths for the entirety of the sample collection head 2620. For example, the width of a first portion (e.g., the proximal end 2622) of the sample collection head 2620 may be different from the width of a second portion (e.g., the distal end 2624) of the sample collection head 2620.

In some embodiments, the sample collection head 2620 (or a portion thereof) includes the same outer and/or inner size (e.g., diameter) for the entirety of the sample collection head 2620. In various embodiments, the sample collection head 2620 includes at least two different sizes (e.g., outer and/or inner diameters) for the entirety of the sample collection head 2620. For example, the size of a first portion of the hollow interior (e.g. at the proximal end 2622) of the sample collection head 2620 may be different from the size of a second portion of the hollow interior (e.g., at the distal end 2624) of the sample collection head 2620.

In some embodiments, the sample collection head 2620, or at least a portion of the sample collection head 2620 tapers (e.g., has sequentially reduced size such as width, outer diameters, and/or inner diameters) towards the proximal end 2622, the distal end 2624, from the middle of the sample collection head 2620 or any point of the sample collection head 2620.

In some embodiments, the sample collection head 2620 tapers from a maximum size at the proximal end 2622 of the sample collection head 2620 to a minimum size at the distal end 2624 of the sample collection head 2620.

In some embodiments, the sample collection head 2620 tapers from a maximum size at the distal end 2624 of the sample collection head 2620 to a minimum size at the proximal end 2622 of the sample collection head 2620.

In some embodiments, the maximum size of the sample collection head 2620 occurs at any point of the sample collection head 2620 and tapers to a minimum size at the proximal end 2622 and/or the distal end 2624 of the sample collection head 2620.

In some embodiments, the minimum size of the sample collection head 2620 occurs at any point of the sample collection head 2620 and taper to a maximum size at the proximal end 2622 and/or the distal end 2624 of the sample collection head 2620.

In some embodiments, the size of the sample collection head 2620 alternates between a minimum size and a maximum size from the proximal end 2622 to the distal end 2624 of the sample collection head 2620.

In general, the sample collection head 2620 can include any structures for collecting biological samples (e.g., cells). In some embodiments, the sample collection head 2620 includes repeating or alternating porous or non-porous structures, such as fins, nubs, bumps, surfaces, bars, cavities formed therefrom, or combinations thereof as shown, for example, in FIGS. 26-33. In general, the structures can be arranged in any desired pattern. In some embodiments, the sample collection head (or a portion thereof) includes a solid interior. In some embodiments, the sample collection head (or a portion thereof) includes an hollow interior.

In some embodiments, the alternating structures of the sample collection head form a cross section that includes one or more symmetric convex and/or concave curves. In some embodiments, the alternating fins, surfaces, and/or cavities form a cross section that includes one or more asymmetric convex curves and/or concave curves. In some embodiments, the cross-section includes the same shape (e.g., V shape, U Shape, circular shape, or any other shapes) for the entirety of the length of the sample collection head. In some embodiments, the cross-section includes at least two different shapes for the entirety of the length of the sample collection head.

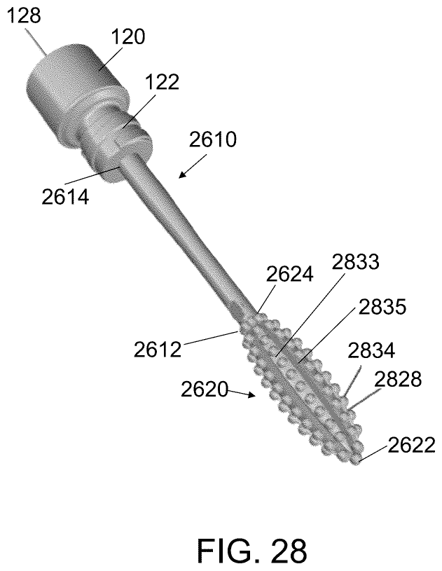

As used herein, a “cavity” or “space” of a sample collection head refers to a hollow portion of the sample collection head that can provide room for scraping, retaining, attracting, holding, and/or storing samples (e.g., biological samples) in any dimension (e.g., 3 dimensions). In general, the cavity can include any regular or irregular shape or size. In some embodiments, the cavity is formed by or between one or more portions of the sample collection head, such as fins, nubs, outer surfaces, bars, porous structures, or combinations thereof. In some embodiments, a cavity is concave, which has a surface that is curved or rounded inward relative to the sample collection device (e.g., nubs in FIGS. 28-30, and 32). In some embodiments, a cavity is convex, which has a surface that is curved or rounded inward relative to the sample collection device (e.g., cavities in FIGS. 26, 27, 29-33). In use, a cavity can provide room or spaces such that the sample collection head can scrape a collection area (e.g., skin) and/or a portion of biological material, where the samples (e.g., blood, semen, saliva, urine, feces, hair, teeth, bone, buccal cells, sores, poxes, tissues, tumors, or pathogens) are retained, held, and collected in the cavity.

In some embodiments, a cavity includes a size or dimension (e.g., thickness, width, outer diameter, and/or inner diameter) in a range from about 0.1 mm to about 6 mm, from about 0.5 mm to about 5.5 mm, from about 1.0 mm to about 5 mm, from about 1.5 mm to about 4.5 mm, from about 2 mm to about 4 mm, from about 2.5 mm to about 3.5 mm, about 1 mm to about 100 mm, about 1 mm to about 75 mm, from about 1 mm to about 50 mm, from about 5 mm to about 45 mm, from about 10 mm to about 40 mm, from about 15 mm to about 35 mm, from about 20 mm to about 30 mm, from about 50 mm to about 100 mm, from about 75 mm to about 100 mm, or from about 50 mm to about 75 mm.

In some embodiments, a cavity includes substantially the same size towards one or both ends of the cavity. In some embodiments, the cavity tapers (e.g., has sequentially reduced dimension or size) towards one or both ends of the cavity, from the middle or any point of the cavity. For example, the cavity tapers from a maximum size at a first end of the cavity to a minimum size at a second end of the cavity. In another example, the maximum size of the cavity occurs at any point of the cavity and the diameters taper to a minimum size at one or both end of the cavity.

Advantageously, the tapered cavity or other tapered structures as described herein can provide at least mechanical flexibility in the collection head, and/or impact the way that the samples are collected (e.g., wicked into the cavity).

In some embodiments, a cavity is porous along at least one dimension. In some embodiments, a cavity is non-porous along at least one dimension. In some embodiments, a cavity is porous in one dimension and non-porous in another dimension.

In some embodiments, a cavity includes one or more (2, 3, 4, 5, or 6) sides in a 3D space. In some embodiments, a cavity includes two sides that are parallel to each other. In some embodiments, a cavity includes two or more sides that intersect with each other.

In some embodiments, a cavity can hold an amount of sample ranging from about 0.1 ml to about 10 ml, about 0.5 ml to about 8 ml, from 1 ml to about 6 ml, from about 2 ml to about 4 ml, or about 3 ml.

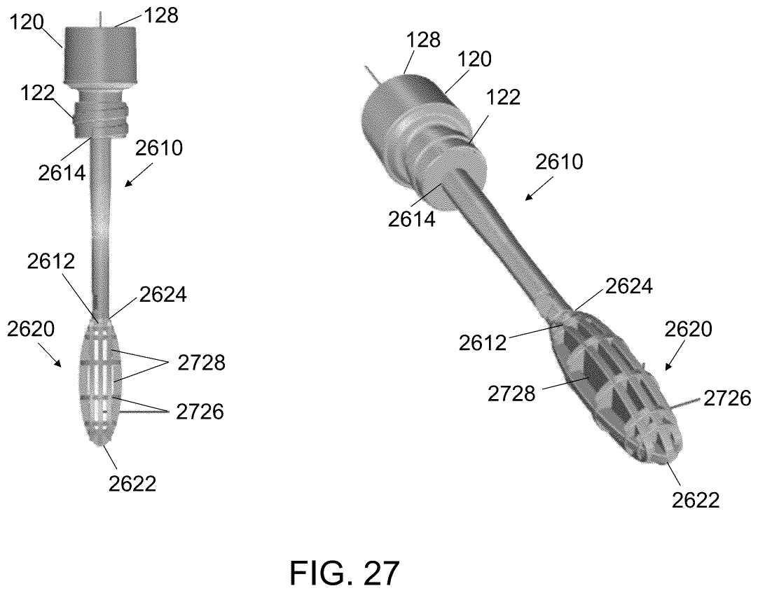

In some embodiments (e.g., FIGS. 26, 29, and 30-33), the sample collection head 2620 (or a portion thereof) includes an angled edge formed by two sides of the sample collection head. In some embodiments (e.g., FIGS. 27 and 28) the sample collection head 2620 (or a portion thereof) includes an edge that is smooth, curved, or round without angles.

Referring to FIG. 26, in some embodiments, the sample collection head 2620 includes one or more (e.g., 1, 2, 3, 4, 5, or more) fins 2626 and cavities 2628, where a cavity is formed by the fin or two adjacent fins. In some embodiments, a fin 2626 is a parallel fin. In some embodiments, a fin 2626 is a non-parallel fin. In some embodiments, a fin 2626 includes substantially the same length as the sample collection head 2620 (e.g., the distance between the proximal end 2622 and the distal end 2624). In some embodiments, a fin 2626 includes any length, e.g., a length that is shorter than the sample collection head 2620 (e.g., the distance between the proximal end 2622 and the distal end 2624).

In some embodiments, a fin 2626 includes a size (e.g., thickness, width, depth, or other types of sizes) in a range from about 0.1 mm to about 6 mm, from about 0.5 mm to about 5.5 mm, from about 1.0 mm to about 5 mm, from about 1.5 mm to about 4.5 mm, from about 2 mm to about 4 mm, from about 2.5 mm to about 3.5 mm, from about 1 mm to about 100 mm, from about 5 mm to about 50 mm, from about 10 mm to about 40 mm, from about 15 mm to about 35 mm, from about 20 mm to about 30 mm, from about 50 mm to about 100 mm, from about 50 mm to about 75 mm, or from about 75 mm to about 100 mm.

In some embodiments, a fin 2626 includes substantially the same size towards one or both ends of the fin 2626. In some embodiments, the fin 2626 tapers (e.g., has sequentially reduced size) towards one or both ends of the fin 2626, from the middle or any point of the fin 2626. In some embodiments, the fin 2626 tapers from a maximum size at a first end of the fin 2626 to a minimum size at a second end of the fin 2626. In another example, the maximum size of the fin 2626 occurs at any point of the fin 2626 and tapers to a minimum size at one or both end of the fin 2626.

Advantageously, the fin can provide improved performance such as increased amounts of collected samples as compared to a traditional sample collection device [(e.g., in scraping, scooping, sucking, or retaining the sample along the sample collection head 2620).

As used herein, a “fin” refers to a membranous, winglike, or paddlelike structure that can be coupled to a cap, a stem, or another portion of a collection device for collecting samples. In some embodiments, one or more fins form a cavity in which the sample can be retained or stored.

Referring still to FIG. 26, in some embodiments, a cavity 2628 includes hollow space formed above a surface of a fin 2626. Accordingly, the sample collection head 2620 may include repeating fins 2626 or cavities 2628 along a direction that is substantially perpendicular to the direction from the proximal end to the distal end of the sample collection head.

In some embodiments, the fins 2626 and cavities 2628 of the sample collection head form a cross section 2650 that includes one or more symmetric convex and/or concave curves. In some embodiments, the alternating fins 2626 and cavities 2628 form a cross section 2650 that includes one or more asymmetric convex and/or concave curves. In some embodiments, the cross-section 2650 includes substantially the same shape (e.g., V shape, U Shape, circular shape, or any other shapes) for the entirety of the length of the sample collection head 2620. In some embodiments, the cross-section 2650 includes at least two different shapes for the entirety of the length of the sample collection head 2620.

Referring to FIGS. 27-28, in some embodiments, the sample collection head 2620 includes a base structure 28 that is similar to a head portion of a regular swab, and one or more nubs, surfaces (e.g., outer surface), and/or cavities formed therefrom on the base structure, as described herein.

In some embodiments, a surface of the sample collection head is flat (e.g., FIG. 27). In some embodiments, a surface of the sample collection head 2620 is uneven (e.g., FIG. 28). In some embodiments, a surface of the sample collection head 2620 includes one or more nubs or any other structures. In some embodiments, a surface of the sample collection head 2620 includes alternating hills and valleys along a circumference of the sample collection head 2620 (e.g., FIG. 28).

In some embodiments, two surfaces are parallel to each other. In some embodiments, two surfaces are interconnecting with each other.

As shown in FIG. 27, in some embodiments, the sample collection head 2620 includes one or more (e.g., 1, 2, 3, 4, 5, or more) surfaces 2726 and cavities 2728 formed between or by two or more surfaces 2726.

In some embodiments, the surface 2726 includes substantially the same length as the sample collection head 2620 (e.g., the distance between the proximal end 2622 and the distal end 2624). In some embodiments, the surface 2726 includes any length, e.g., a shorter length than the sample collection head 2620 (e.g., the distance between the proximal end 2622 and the distal end 2624).

In some embodiments, the surface 2726 includes a size (e.g., width, height, depth, thickness, or other types of sizes) in a range from about 0.1 mm to about 6 mm, from about 0.5 mm to about 5.5 mm, from about 1.0 mm to about 5 mm, from about 1.5 mm to about 4.5 mm, from about 2 mm to about 4 mm, from about 2.5 mm to about 3.5 mm, from about 1 mm to about 100 mm, from about 5 mm to about 50 mm, from about 10 mm to about 40 mm, from about 15 mm to about 35 mm, from about 20 mm to about 30 mm, from about 50 mm to about 100 mm, from about 50 mm to about 75 mm, or from about 75 mm to about 100 mm.

In some embodiments, the surface 2726 includes substantially the same thickness, width, outer diameter, and/or inner diameters towards one or both ends of the surface 2726. In some embodiments, the surface 2726 tapers (e.g., has sequentially reduced thickness, width, outer diameters, and/or inner diameters) towards one or both ends of the surface 2726, from the middle or any point of surface 2726. For example, the surface 2726 tapers from a maximum diameter at a first end of the surface 2726 to a minimum diameter at a second end of the surface 2726. In another example, the maximum diameter of the surface 2726 occurs at any point of the surface 2726 and the diameters taper to a minimum diameter at one or both end of the surface 2726.

Advantageously, the surfaces can provide improved performance in wicking, scraping, directing the direction in which sample is stored and retained, retaining, and/or attracting sample along the sample collection head 2620, as compared with traditional sample collection devices.

In some embodiments, at least two surfaces 2726 are substantially parallel to each other. In some embodiments, at least two surfaces 2726 are substantially vertical to each other, and thus form a pair of vertical angles that are opposite angles with a common vertex (e.g., the point of intersection). In some embodiments, two of the surfaces 2726 are intersecting in a three dimensional space. In some embodiments, two of the surfaces 2726 cross each other at any angle between 0 and 180°. In some embodiments, two of the surfaces are substantially parallel to each other, which are vertical or cross at any angle with one or more of other surfaces.

In some embodiments, a cavity 2728 includes hollow space between or by at least two surfaces 2726 to collect or hold a sample. As shown in FIG. 27, the sample collection head 2620 can include repeating or alternating surfaces 2726 and cavities 2628. In some embodiments, the alternating surfaces 2726 and cavities 2728 form a cross section that includes one or more symmetric convex and/or concave curves. In some embodiments, the alternating surfaces 2726 and cavities 2728 form a cross section that includes one or more asymmetric convex and/or concave curves. In some embodiments, the cross-section includes substantially the same shape for the entirety of the length of the sample collection head 2620. In some embodiments, the cross-section includes at least two different shapes for the entirety of the length of the sample collection head 2620.

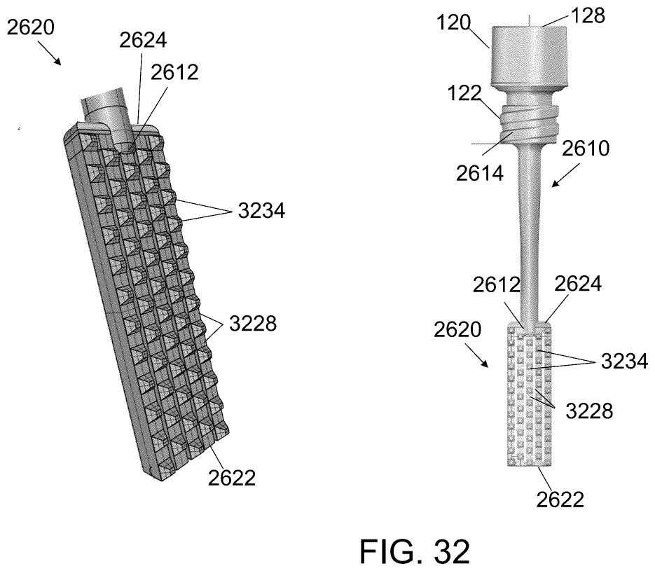

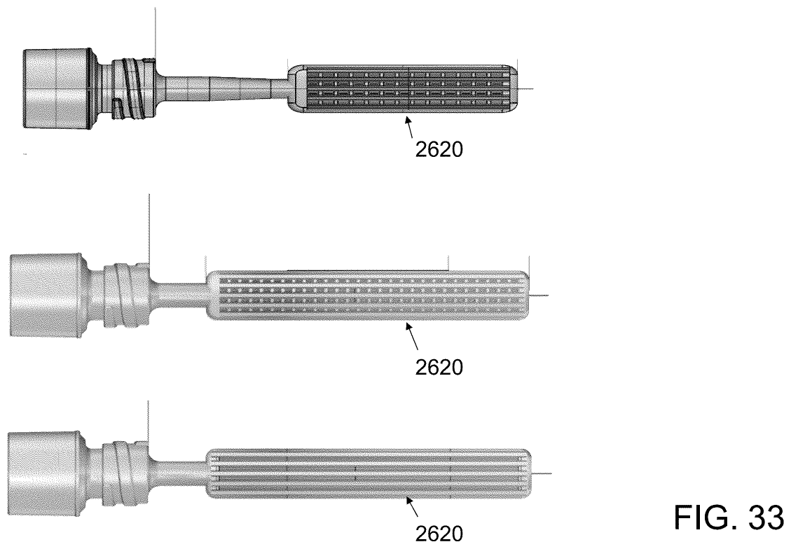

Referring to FIG. 28, in some embodiments, the sample collection head 2620 includes one or more (e.g., 1, 2, 3, 4, 5, 10, 15, 20, or more) nubs 2834 and cavities 2828 formed between or by the nubs 2726. In some embodiments, a nub 2827 includes any shape, such as hemisphere-shape (FIG. 28) or a pyramid-shape (FIG. 32). In some embodiments, the nubs 2827 include substantially the same shape for the entirety of the length of the sample collection head 2620. In some embodiments, the nubs 2827 include at least two different shapes for the entirety of the length of the sample collection head 2620.

As used herein, a “nub” or “bump” refers to a protuberance, a protrusion, a lump, or a small piece that is generated on the surface of a collection device. In some embodiments, a cavity or a space can be formed between two nubs for collecting, retaining, or storing samples.

In some embodiments, the nub 2827 includes a size (e.g., thickness, width, height, outer diameter, or other types of sizes) in a range from about 0.05 mm to about 6 mm, from about 0.5 mm to about 5.5 mm, from about 1.0 mm to about 5 mm, from about 1.5 mm to about 4.5 mm, from about 2 mm to about 4 mm, from about 2.5 mm to about 3.5 mm, from about 1 mm to about 100 mm, from about 5 mm to about 50 mm, from about 10 mm to about 40 mm, from about 15 mm to about 35 mm, from about 20 mm to about 30 mm, from about 50 mm to about 100 mm, from about 50 mm to about 75 mm, or from 75 mm to about 100 mm.

In some embodiments, the nub 2827 includes substantially the same size towards one or both ends of the nub 2827. In some embodiments, the nub 2827 tapers (e.g., has sequentially reduced size) towards one or both ends of the nub 2827, from the middle or any point of nub 2827. For example, the nubs 2827 taper from a maximum size at a first end (e.g., proximal end 2622) of the sample collection head 2620 to a minimum size at a second end (e.g., distal end 2624) of the sample collection head 2620. In another example, the maximum size of the nub 2827 occurs at any point of the sample collection head 2620 and tapers to a minimum size at one or both proximal end and distal end of the sample collection head 2620.

Advantageously, the nub 2827 along the sample collection head 2620 can abrase and/or scarify a sample collection area (e.g., a skin area) without using an additional tool.

Additionally, the nub 2827 can provide improved performance in sample collection such as scraping, retaining and attracting samples, as compared to a traditional sample collection device.

In some embodiments, the sample collection head 2620 further includes one or more protruded surfaces 2833 and valleys 2835 formed between the protruded surfaces 2833. In some embodiments, one or more nubs 2827 are located at the protruded surface 2833. In some embodiments, one or more nubs 2827 are located at the valley 2835. In some embodiments, the nubs 2827 are located along one or more lines between the distal end 2624 and the proximal end 2622 at the outer surface of the sample collection head 2620.

In some embodiments, two adjacent nubs include substantially the same distance for the entirety of the length of the sample collection head 2620. In some embodiments, two adjacent nubs include at least two different distances for the entirety of the length of the sample collection head 2620.

Referring still to FIG. 28, in some embodiments, a cavity 2828 includes hollow space formed between or by the nubs 2827, protruded surfaces 2833, and/or valleys 2835.

Accordingly, the sample collection head 2620 can include alternating nubs 2834 and cavities 2828.

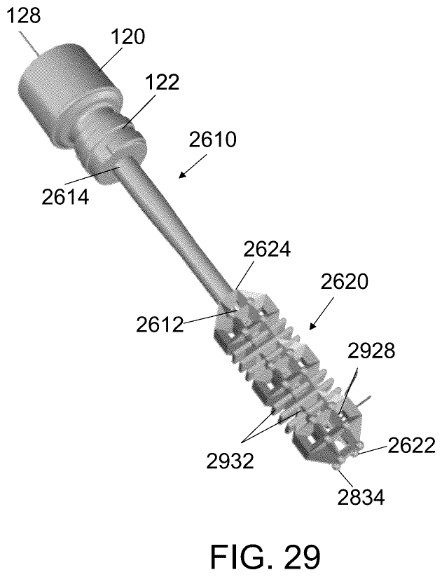

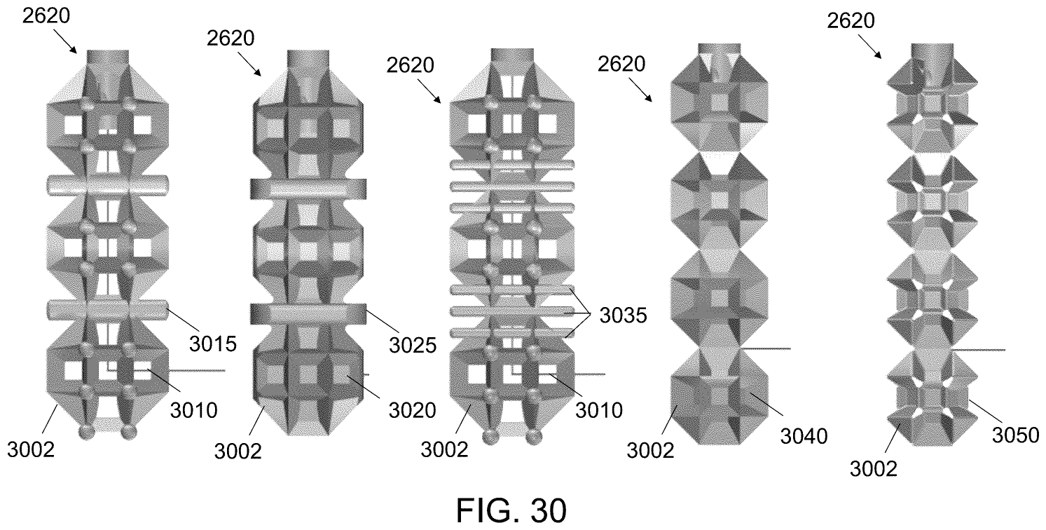

FIGS. 29 and 30 illustrates collection devices including exemplary sample collection heads that include one or more porous structures.

As used herein, a “porous” or “pore” refers to a structure that includes pores, holes, or cavities of any shape, through which air or fluid can flow. In some embodiments, a “pore” is equivalent to a “cavity” as used herein.

Referring to FIG. 29, in some embodiments, the sample collection head 2620 includes a porous structure that includes one or more (e.g., 1, 2, 3, 4, 5, 10, 15, or more) bars 2932 and cavities 2928 formed between or by the bars 2932. In some embodiments, a bar 2932 includes one or more flat surfaces. In some embodiments, a bar 2932 includes one or more uneven surfaces.

In some embodiments, a bar 2932 includes substantially the same length as the sample collection head 2620 (e.g., the distance between the proximal end 2622 and the distal end 2624). In some embodiments, a bar 2932 includes any length, e.g., a length that is shorter than the sample collection head 2620 (e.g., the distance between the proximal end 2622 and the distal end 2624).

In some embodiments, a bar 2932 includes a size (e.g., width, height, depth, thickness, or other types of sizes) in a range from about 0.1 mm to about 6 mm, from about 0.5 mm to about 5.5 mm, from about 1.0 mm to about 5 mm, from about 1.5 mm to about 4.5 mm, from about 2 mm to about 4 mm, from about 2.5 mm to about 3.5 mm, from about 1 mm to about 100 mm, from about 5 mm to about 50 mm, from about 10 mm to about 40 mm, from about 15 mm to about 35 mm, from about 20 mm to about 30 mm, from about 50 mm to about 100 mm, from about 50 mm to 75 mm, or from about 75 mm to about 100 mm.

In some embodiments, a bar 2932 includes substantially the same size towards one or both ends of the bar. In some embodiments, a bar 2932 tapers (e.g., has sequentially reduced size) towards one or both ends of the bar, from the middle or any point of the bar. For example, a bar tapers from a maximum size at a first end to a minimum diameter at a second end of the bar. In another example, the maximum size of a bar occurs at any point of the bar and tapers to a minimum size at one or both end of the bar. In some embodiments, the bar provides improved performance as compared with traditional sample collection devices, by, including creating friction to loosen the cells, tissues, biological material at a location and increasing surface area between the bars to collect and store the biological material along the sample collection head 2620.

In some embodiments, at least two bars 2932 are substantially parallel to each other. In some embodiments, at least two bars 2932 are substantially vertical to each other, and thus form a pair of vertical angles that are opposite angles with a common vertex (e.g., the point of intersection). In some embodiments, at least two bars 2932 are intersecting in a three dimensional space. In some embodiments, at least two bars 2932 cross each other at any angle between 0 and 180°. In some embodiments, at least two bars 2932 are substantially parallel to each other, which are vertical or cross at any angle with one or more of other surfaces.

Referring still to FIG. 29, in some embodiments, the cavities 2928 includes hollow space between or by at least two bars 2932. Accordingly, the sample collection head 2620 can include alternating bars 2932 and cavities 2928.

In some embodiments, the sample collection head 2620 further includes one or more nubs 2834 at the proximal end 2622. In some embodiments, a nub 2834 includes a hemisphere shape, or any other shapes.

In general, the sample collection head 2620 can include any repeating or alternating structures as described herein, such as fins, surfaces, nubs, bars, cavities or pores formed therefrom, or combinations thereof.

As shown in FIG. 30, in some embodiments, the sample collection head 2620 includes repeating polygonal three dimensional (3D) structures 3002. In some embodiments, the polygonal three dimensional (3D) structures include one or more cavities that are porous and/or non-porous. In some embodiment, a cavity 3010 or 3050 is porous in one dimension and non-porous in another dimension. In some embodiments, a cavity 3010 includes four sides. In some embodiments, a cavity 3010 includes two sides that are parallel to each other along a first dimension, and two sides that are parallel to each other along a second dimension. In some embodiments, a cavity 3020 further includes an additional side that is perpendicular to a third dimension that is different from the first and the second dimensions.

In some embodiments, the sample collection head 2620 further includes one or more (e.g., 1, 2, 3, 4, 5, or more) bars between two adjacent polygonal three dimensional (3D) structures. In some embodiments, a bar 3015 or 3035 has one or more round edges. In some embodiments, a bar 3025 has an angled edge.

In some embodiments, the sample collection head 2620 further includes one or more (e.g., 1, 2, 3, 4, 5, or more) nubs. In some embodiments, the nubs are located at the intersection points of two bars.

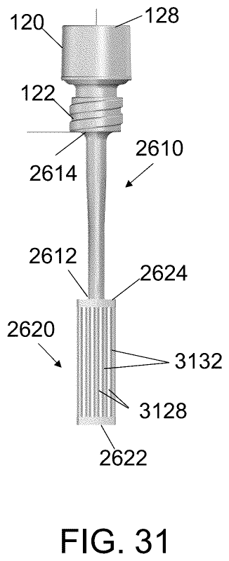

Referring to FIG. 31, in some embodiments, the sample collection head 2620 includes one or more (e.g., 1, 2, 3, 4, 5, or more) bars 3132 and cavities 3128 formed as an open space between or by the bars 3132.

In some embodiments, a bar 3132 includes substantially the same length as the sample collection head 2620 (e.g., the distance between the proximal end 2622 and the distal end 2624). In some embodiments, the bar 3132 includes any length, e.g., a length that is shorter than the sample collection head 2620 (e.g., the distance between the proximal end 2622 and the distal end 2624).

In some embodiments, the bar 3132 includes a size (e.g., width, height, depth, thickness, or other types of sizes) in a range from about 0.1 mm to about 6 mm, from about 0.5 mm to about 5.5 mm, from about 1.0 mm to about 5 mm, from about 1.5 mm to about 4.5 mm, from about 2 mm to about 4 mm, from about 2.5 mm to about 3.5 mm, from about 1 mm to about 75 mm, from about 5 mm to about 50 mm, from about 10 mm to about 40 mm, from about 15 mm to about 35 mm, from about 20 mm to about 30 mm.

In some embodiments, the bar 3132 includes substantially the same size towards one or both ends of the bar 3132. In some embodiments, the bar 3132 tapers (e.g., has sequentially reduced size) towards one or both ends of the bar 3132, from the middle or any point of the bar 3132. For example, the bar 3132 tapers from a maximum size at a first end of the bar 3132 to a minimum size at a second end of the bar 3132. In another example, the maximum size of the bar 3132 occurs at any point of the fin bar 3132 and tapers to a minimum size at one or both end of the bar 3132. Advantageously, the bar provides improved performance as compared with traditional sample collection devices, including creating friction to loosen the cells, tissues, biological material at a location and increases surface area between the bars to collect and store the biological material along the sample collection head 2620.

In some embodiments, at least two bars 3132 are substantially parallel to each other. In some embodiments, at least two bars 3132 are not parallel to each other. In some embodiments, at least two bars 3132 are substantially vertical to each other, and thus form a pair of vertical angles that are opposite angles with a common vertex (e.g., the point of intersection). In some embodiments, at least two bars 3132 are intersecting in a three dimensional space. In some embodiments, at least two bars 3132 cross each other at any angle between 0 and 180°. In some embodiments, at least two bars 3132 are vertical or cross at any angle with one or more of other surfaces.

As used herein, a “bar” refers to a solid structure in the form of an oblong or a rectangle.

Referring still to FIG. 31, in some embodiments, a cavity 3128 is formed as hollow space between or by two adjacent bars 3132. Accordingly, the sample collection head 2620 can include alternating bars 3132 and cavities 3128.

Referring to FIG. 32, in some embodiments, the sample collection head 2620 includes one or more (e.g., 1, 2, 3, 4, 5, or more) bumps formed as series along lines between two ends of the sample collection head 2620. The bumps can include any structure, such as a pyramid structure 3234. In some embodiments, the sample collection head 2620 further includes one or more cavities 3228 formed between or by at least two bumps. In some embodiments, the bumps are configured at the sample collection head 2620 such that bumps 3234 and cavities 3228 are alternating between the two ends and/or between the two sides of the sample collection head 2620.

In some embodiments, a bump 3234 includes a size (thickness, width, height, outer diameter, inner diameters, or other types of sizes) in a range from about 0.1 mm to about 6 mm, about 0.5 mm to about 5.5 mm, about 1.0 mm to about 5 mm, about 1.5 mm to about 4.5 mm, about 2 mm to about 4 mm, about 2.5 mm to about 3.5 mm, about 1 mm to about 50 mm, about 5 mm to about 45 mm, about 10 mm to about 40 mm, about 15 mm to about 35 mm, about 20 mm to about 30 mm.

In some embodiments, a bump 3234 includes substantially the same size towards one or both ends of the sample collection head 3220. In some embodiments, a bump 3234 tapers (e.g., has sequentially reduced size) towards one or both ends of the sample collection head 2620, from the middle or any point of the sample collection head 2620.

Referring still to FIG. 32, in some embodiments, a cavity 3228 is formed as hollow spaces between or by at least two bumps 3234. Accordingly, the sample collection head 2620 can include alternating bars 3134 and cavities 3228.

In use, the series of bumps 3234 can provide room or spaces between the bumps 3234, i.e., the cavities 3228, such that the sample collection head can scrape and retain samples that are collected.

FIG. 33 illustrates example sample collection heads with various length. In general, the sample collection head can include any length, such as the ranges described above. In particular embodiments, the sample collection head 2620 includes a length of about 22.5 mm or about 30 mm. In some embodiments, a different length is selected depending on the condition (e.g., contact area, moist condition. In some embodiments, the sample collection head is configured to contact a collection area (e.g., skin) ranging from about 10 mm² to about 1000 mm², from about 50 mm² to about 900 mm², from about 100 mm² to about 800 mm², from about 150 mm² to about 700 mm², from about 200 mm² to about 600 mm², from about 250 mm² to about 500 mm², from about 300 mm² to about 400 mm², or about 350 mm².

Materials for Manufacturing a Collection Device

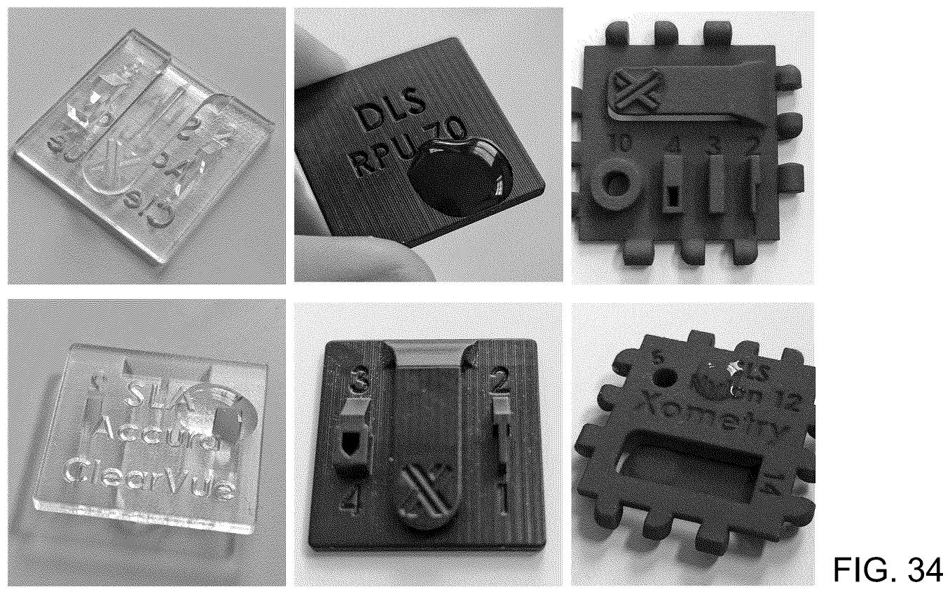

FIG. 34 are images of embodiments of materials used for manufacturing a collection device. In some embodiments, at least some of the materials used for manufacturing a collection device, as described herein, can be used for manufacturing a vial.

In some embodiments, one or more materials can be used to manufacture at least one component of a collection device (e.g., capillary, cap, or their components), according to various embodiments.

In some embodiments, the collection device material exhibits at least one of the following characteristics: (1) sufficiently rigid for collection of samples; (2) sufficiently flexible for safety of use; (3) collects adequate sample for subsequent tests; (4) withstands the rigors of sterilization/disinfection without structural weakening, or chemically interfering subsequent testing. (5) compatible with subsequent testing (e.g., PCR testing and/or nucleic acid extraction technologies, antigen testing, antibody-based testing).

In some embodiments, the collection device material is biodegradable. In some embodiments, the biodegradable collection device materials include a biobased plastic, polyhydroxyalkanoate (PHA), polylactic acid (PLA), starch blend, cellulose-based plastic, lignin-based polymer composite, a petroleum-based plastic, polyglycolic acid (PGA), polybutylene succinate (PBS), polycaprolactone (PCL), poly(vinyl alcohol) (PVA, PVOH), polybutylene adipate terephthalate (PBAT), polyethylene terephthalate glycol (PETG), polypropylene (PP), Pebax, gelatin, collagen, chitosan, starch, sucrose, soy based materials, or other natural-sugar based materials, materials incorporate enzymatically labile groups, and/or their derivatives or combinations.

In some embodiments, the collection device material is water-soluble. In some embodiments, e.g., after the contacting step, at least a portion of the water-soluble collection device (e.g., the soluble portion) is dissolved, e.g., with water or an aqueous solution. Such a dissolving step can permit faster release of the sample from the collection device for downstream applications. In some embodiments, the dissolving step can implement a colorimetric change or other diagnostic detection methods.

In some embodiments, the collection device material is a polymer. In some embodiments, the collection device material includes at least one of polypropylene, polycarbonate, thermoplastic elastomers (TPE), rubber, polyester fiber, acrylonitrile butadiene styrene (ABS), acrylic, polyetherimide, ionomer, acetal copolymer, polyurethane, polystyrene, nylon, Pebax, 3847MR polypropylene, Flint Hills 11R12A homopolymer, zeonex/zeonor or any combination thereof.

In some embodiments, the collection device material is a 3D printed material, including BASF Polypropylene Ultrasint natural polypropylene, nylon (PA 11, PA12, other grades), Accura resin, Somos WaterClear® Ultra 10122, thermoplastic resins, laser sintered metals, and others. In some embodiments, the collection device material is a compression molded powder, rubber, pellet, or other.

In some embodiments, the collection device is comprised of multiple materials manufactured through differing processes, including compression molding, injection molding, extrusion, 3D printing and others.

In some embodiments the collection device material includes polyvinyl alcohol or a derivative polymer such as polyvinyl acetals, polyvinyl butyral (PVB), or polyvinyl formal (PVF). In some embodiments, the collection device material comprises Kuraray MOWIFLEX™ C17 or C30 materials, which are PVA variants. In some embodiments, the collection device material comprises Kuraray POVAL™ materials.

In some embodiments, the collection device material is glass.

In some embodiments, the collection device material is plastic.

In some embodiments the material is a cast urethane.

In some embodiments, the collection device material is a solid material (i.e., non-porous).

In some embodiments the collection device material is flocked/fibrous.

In some embodiments the collection device material is hydrophilic.

In some embodiments, the collection device material is a foam.

In some embodiments, the collection device material includes a paper (e.g., FTA paper Whatman 903, Ahlstrom 226, or other) or cellulose, bamboo, cotton or other pulp substrate.

In some embodiments, the collection device material is a filter, filter paper, or combination/layering of multiple webs.

In some embodiments, the collection device material is nonwoven or other web-based material.

In some embodiments, the collection device material includes bicomponent fibers. [00284] In some embodiments, the collection device material includes cotton or other natural fibers.

In some embodiments, the collection device material includes synthetic fibers, including glass, nylon, and others.

In some embodiments, the collection device material includes engineered films with hydrophilicity or other material properties.

In some embodiments, the collection device material is hydrophobic. In some embodiments, the collection device material is a porous material. In some embodiments, the collection device material is medical grade. In some embodiments, the collection device material exhibits the following features: autoclave sterilizable; E-beam sterilizable; ethylene oxide sterilizable; no animal derived components; and radiation sterilizable

In some embodiments, the collection device material can withstand surface treatments such as chemical treatments, surface modifications (e.g., plasma treatment), and/or mechanical treatments (e.g., surface texturing, cross-hatching, or high polish), or any other treatments.

In some embodiments, the collection device material has a flexural modulus (also referred to as bending modulus, which is the ratio of stress to strain in flexural deformation, or the tendency for a material to resist bending) of about 100 megapascals (MPa) to 5000 MPa.

In some embodiments the collection device consists of any combination of the materials listed above.

Manufacture of a Collection Device

In some embodiments, at least one component of the collection device (e.g., capillary, cap or their components as described herein) is manufactured using molding (e.g., injection molding, or overmolding), stamping, die cutting, thermal cutting, ultrasonic welding, extrusion, milling, lamination, compression molding, or 3D printing. For example, at least one component of the collection device (e.g., capillary, cap or their components as described herein) is configured to be ejected from a mold.

In some embodiments, at least one component of the collection device (e.g., capillary, cap or their components as described herein) is manufactured so as to not result in abrasive or sharp features, in order to avoid damage to the subject and ensure a safe use of the collection device.

In some embodiments, at least one component of the collection device 100 is fabricated from at least one polymer (e.g, polypropylene, and/or other suitable polymer materials). In some embodiments, at least one component of the collection device is fabricated from glass.

In some embodiments, a method of manufacturing the at least one component of a collection device includes: 1) manufacturing a mold, e.g., according to the dimensions and/or features of the collection device; 2) injecting a mold with a liquid form of one or more collection device material(s); and/or 3) removing at least a portion of the collection device (e.g., the capillary) from the mold once solidified.

In some embodiments, components of the device may be manufactured through differing methods, then assembled together either permanently, or removably. For example, a

3D printed component may be combined with an injection molded material, then assembled through heat-staking. Or an insert (paper, glass fiber, foam etc) may be assembled into an injection molded component and held in place via press-fit.

Referring to FIG. 25, in some embodiments, the method of manufacturing of the collection device includes injection molding. In particular embodiments, the method of manufacturing of the collection device includes a two-shot injection molding, where a first shot includes manufacturing cap and mechanical feature to hold the material manufactured in a second shot. In some embodiments, the second shot material can be determined to achieve desired sample collection performance. In some embodiments, examples of materials used in second shot are selected from hydrophilic polymers, foams, soluble collection mediums, or any other suitable materials.

In some embodiments, the collection device material(s) is liquefied, e.g., at a temperature of about 420° C.

In some embodiments, at least one component of the collection device may be molded separately.

In some embodiments, at least part of the collection device may be configured to be ejected from a mold. In some embodiments, at least part of the collection device may be configured (e.g., machined) to include a non-abrasive feature instead of abrasive or sharp features, in order to avoid damage to the subject and ensure a safe use of the collection device.

Vial

In some embodiments, the threaded portion 122 of the cap 120 is configured to seal with and/or interlock with the opening of a vial. For example, after the sample is collected, the threaded portion (e.g., external or internal threads) of the collection device may be screwed to the threaded portion of the vial.

In some embodiments, the cap 120 and/or the vial may include an external and/or internal structure (e.g., O-ring or gasket) to aid in forming a substantially fluid-tight seal between the cap 120 and the vial and in stopping liquid from leaking from the vial.

In some embodiments, the maximum diameter of the cap 120 is greater than, or substantially the same as the diameter of the opening of the vial.

In some embodiments, the vial contains sample transport media. In other embodiments, the vial is dry before in use.

In some embodiments, the vial can be constructed from a transparent material.

In some embodiments, the vial includes a length (e.g., a distance between the opening of the vial and the bottom of the vial) that is substantially the same as or greater than the length of the shortened capillary of the collection device.

In some embodiments, the vial has an inner diameter that is greater than the maximum diameter of the capillary inserted in the vial.

In some embodiments, the vial includes a threaded portion at or in proximity to the opening of the vial. In some embodiments, the threaded portion of the vial includes at least 1, 2, 3, 4, or 5 threads (e.g., internal and/or external threads) to allow the cap 120 to be screw onto the vial. In some embodiments, the threads are continuous and/or discontinuous.

In some embodiments, the vial is compatible for use with an automated device. In some embodiments, the vial is compatible with the Society for Biomedical Sciences (SBS) 24-well format, the SBS 48-well format, the SBS 96-well format, or any combination thereof. In some embodiments, the vial is compatible for use with universal/large format 12, 24, 36, 48 tube formats.

In some embodiments, the vial (e.g., vials in FIGS. 4-6) includes a volume from about 1 ml to about 10 ml. In some embodiments, the vial includes a volume from about 0.1 ml to about 20 ml. In some embodiments, the vial includes a volume from about 1 ml to about 8 ml. In some embodiments, the vial includes a volume from about 3 ml to about 6 ml. In preferred embodiments, the vial includes a volume from about 4 ml to about 5 ml. In preferred embodiments, the vial includes a volume of about 4 ml, 4.5 ml, or 5 ml.

In some embodiments, the vial includes a barcode at the outside surface of the vial.

In some embodiments, the vial includes an average width from about 10 mm to about 100 mm, about 15 mm to about 95 mm, from about 20 mm to about 90 mm, from about 25 mm to about 85 mm, from about 30 mm to about 80 mm, from about 35 mm to about 75 mm, from about 40 mm to about 70 mm, from about 45 mm to about 65 mm, from about 50 mm to about 60 mm. In some embodiments, the vial includes an average width of about any of: 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm. In some embodiments, the vial includes an average width of at least about any of: 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm. In some embodiments, the vial includes an average width of at most about any of: 10 mm, 11 mm, 12 mm, 13 mm, 14 mm, 15 mm, 16 mm, 17 mm, 18 mm, 19 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm.

In some embodiments, the vial includes a height from about 10 mm to 100 mm, about 15 mm to about 95 mm, about 20 mm to about 90 mm, about 25 mm to about 85 mm, about 30 mm to about 80 mm, about 35 mm to about 75 mm, about 40 mm to about 70 mm, about 45 mm to about 65 mm, or about 50 mm to about 60 mm, about 50 mm to about 55. In some embodiments, the vial includes an average width of about any of: 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm. In some embodiments, the vial includes an average width of at least about any of: 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm. In some embodiments, the vial includes an average width of at most about any of: 10 mm, 15 mm, 20 mm, 25 mm, 30 mm, 35 mm, 40 mm, 45 mm, 50 mm, 55 mm, 60 mm, 65 mm, 70 mm, 75 mm, 80 mm, 85 mm, 90 mm, 95 mm, or 100 mm.

In some embodiments, the vial is coupled to the cap. Such vial-cap assembly includes a height from about 10 mm to about 120 mm, about 20 mm to about 110 mm, about 30 mm to about 100 mm, about 40 mm to about 90 mm, about 50 mm to about 80 mm, about 60 mm to about 70 mm. In some embodiments, the vial-cap assembly includes a height of about any of: 10 mm, 20 mm, 30 mm, 40 mm, 50 mm, 60 mm, 70 mm, 80 mm, 90 mm, 100 mm, 110 mm, or 120 mm. In some embodiments, the vial-cap assembly includes a height of at least about any of: 10 mm, 20 mm, 30 mm, 40 mm, 50 mm, 60 mm, 70 mm, 80 mm, 90 mm, 100 mm, 110 mm, or 120 mm. In some embodiments, the vial-cap assembly includes a height of at most about any of: 10 mm, 20 mm, 30 mm, 40 mm, 50 mm, 60 mm, 70 mm, 80 mm, 90 mm, 100 mm, 110 mm, or 120 mm.

In some embodiments, the vial can include a cross-section of at least one shape of a polygonal shape, e.g., a triangle, a square, a quadrilateral, a trapezoid, a pentagon, a hexagon, a polygon with at least 7, 8, 9, 10, 11, 12, 13, 14, 15, 16, 17, 18, 19, 20 or more sides, a circle, a circle with one or more flat sides, an ellipse, or an ellipse with one or more flat sides. In some embodiments, at least one side of the cross-section includes a convex and/or concave curve. In some embodiments, the cross-section includes a rotationally symmetric shape or an asymmetric shape. In some embodiments, the cross-section includes the same shape, or at least two different shapes for the entirety of the component.

In some embodiments, the collection tube and device are 12-14 mm in diameter and 72-78 mm in height, designed to fit into decapper and diagnostic system (molecular or other) such as but not limited to the Abbott m2000 and Abbott Alinity as well as systems from BD, Beckman Coulter, Hologic, Roche Cobas, and others.

In some embodiments, the vial is automation compatible and can interface with an automation device (e.g., a decapper machine).

In some embodiments, the decapper machine is selected from any one of Rhinostics RHINObot™, Rhinostics ELEbot™, AltemisLab Advantage 96 and other decappers, Altecap Switch, IntelliXcap, Capit-All, Screw Cap CS700, Univo SR048, SafeCap, or other similar instruments from Altemis, Rhinostics, LVL, Hamilton, ThermoFisher, Azenta, Micronics or other manufacturers. In particular embodiments, the decapper machine is manufactured or sold by Rhinostics or other companies.

In some embodiments, the decapper machine is attached to and works with a liquid handler. In some embodiments, the liquid handle is selected from any one of Hamilton, Tecan, Open-trons, Integra, Beckman Coulter, ThermoFisher, or any other manufacturers.

In some embodiments, the vial is selected from any one of Corning, Nest, AltemisLab, Altertube, Azenta Cryotube, Thermo-Nunc, Thermo-Matrix, Micronic, Ziath Cryzotraq, Globe CryoClear, LVL, Thermo-Nalgene, United Scientific, Cryovial, or Labforce (Thomas). In particular embodiments, the vial is manufactured by Rhinostics or other companies.

In some embodiments, the vial is compatible with a rack (e.g., SBS 24-well format, the SBS 48-well format, the SBS 96-well format, and/or other suitable formats) as described in further detail below.

In some embodiments the vial is a universal size (ELEtube) designed to work with the ELEbot decapper for tubes approximately 13×75 mm in dimensions. These vials are a universal size to be compatible with Roche, Abbott, Hologic, Beckman, and other systems.

Add in some cases the tube is coated with EDTA, heparin, boric acid, antibodies, other materials.

In some embodiments, the tube includes a desiccant or material to promote drying of the sample collected.

In some embodiments, an element (e.g., an elastomeric disk) may be used for sealing the cap and vial. In some embodiments, the element extends inward to the capillary for sealing and holding the components of the shortened collection device in place during decapping.

In some embodiments, the vial includes a volume from about 1 ml to about 10 ml. In some embodiments, the vial includes a volume from about 0.1 ml to about 20 ml. In some embodiments, the vial includes a volume from about 1 ml to about 8 ml. In some embodiments, the vial includes a volume from about 3 ml to about 6 ml. In preferred embodiments, the vial includes a volume from about 4 ml to about 5 ml. In preferred embodiments, the vial includes a volume of about 4 ml, 4.5 ml, or 5 ml.

In some embodiments, the vial includes a barcode at the outside surface of the vial.

In some embodiments, the vial includes an average width from about 10 mm to about 15 mm. In some embodiments, the vial includes an average width from about 11 mm to 14

mm. In preferred embodiments, the vial includes an average width from about 12 mm to 13 mm. In preferred embodiments, the vial includes an average width of about 12, 12.1, 12.2, 12.3, 12.4, 12.5, 12.6, 12.7, 12.8, 12.9 or 13.0 mm. In some embodiments, the vial includes an average width of about 0.5 mm.

In some embodiments, the vial includes a height from about 1 mm to 100 mm. In some embodiments, the vial includes a height from about 50 mm to 100 mm. In some embodiments, the vial includes a height from about 60 mm to 70 mm. In some embodiments, the vial includes a height from about 1 mm to 50 mm. In some embodiments, the vial includes a height from about 1 mm to 10 mm. In some embodiments, the vial includes a height of about 1, 2, 3, 4, 5, 6, 7, 8, 9, 10, 20, 30, 40, 50, 60, 70, 80, 90, or 100 mm.

In some embodiments, the vial is coupled to the cap. Such vial-cap assembly includes a height from about 1 mm to 120 mm. In some embodiments, the vial-cap assembly includes a height from about 50 mm to 120 mm. In some embodiments, the vial-cap assembly includes a height from about 50 mm to 90 mm. In some embodiments, the vial-cap assembly includes a height from about 1 mm to 50 mm. In some embodiments, the vial-cap assembly includes a height from about 1 mm to 10 mm. In some embodiments, the vial-cap assembly includes a height of about 1, 2, 3, 4, 5, 6, 7, 8, 9, 10, 20, 30, 40, 50, 60, 70, 80, 90, 100, 110, or 120 mm.

In some embodiments, the vial is automation compatible and can interface with an automation device (e.g., a decapper machine).

In some embodiments, the vial is compatible with a rack (e.g., SBS 24-well format, the SBS 48-well format, the SBS 96-well format, and/or other suitable formats) as described in further detail below.

In some embodiments, an element (e.g., an elastomeric disk) may be used for sealing the cap and vial.

In some embodiments the vial may be sealed to ensure cleanliness with foil, a plug, paper, or other material.

In some embodiments, the element extends inward to the capillary for sealing and holding the components of the shortened collection device in place during decapping.

Kits

In some embodiments, one or more kits (e.g., sample collection kit or test kit) may be used for collecting samples using the collection devices (e.g., collection device 100) as described herein.

In some embodiments, the kit includes 1, 2, 3, 4, 5, 6, 7, 8, 9, 10, 11, 12, 13, 14,

15, 16, 17, 18, 19, 20, 30, 40, 50, 60, 70, 80, 90, 100 or more collection devices and/or vials.

In some embodiments, the kit includes an effective amount of sample transport media. In some embodiments, the sample transport media is supplied in a lyophilized or dried form, a concentrated liquid form that can diluted or suspended in liquid prior to use with the collection device, or a liquid solution (e.g., an aqueous solution, a sterile aqueous solution).

Preferred formulations include those that are non-toxic to the samples (e.g., cells bacteria, viruses) and/or does not affect growth rate or viability. The sample transport media can be supplied in aliquots or in unit doses. In some embodiments, transport media preserves the sample components (e.g., cellular, bacterial, or viral nucleic acids, proteins, antibodies or polypeptides) nucleic acid between the time of sample collection and downstream applications.

In some embodiments, the sample transport media comprises a viral transport media (VTM). The constituents of suitable viral transport media are designed to provide an isotonic solution containing protective protein, antibiotics to control microbial contamination, and one or more buffers to control the pH. Isotonicity, however, is not an absolute requirement; some highly successful transport media contain hypertonic solutions of sucrose. Liquid transport media are used primarily for transporting collection devices or materials released into the medium from a collection device. Liquid media may be added to other specimens when inactivation of the viral agent is likely and when the resultant dilution is acceptable.

In some embodiments, the kit can optionally include one or more agents that permit the detection of cellular, bacterial, or viral nucleic acids or polypeptides in the sample (e.g., test strips).

In some embodiments, the kit optionally comprises informational material. The informational material can be descriptive, instructional, marketing or other material that relates to the methods described herein.

In some embodiments, the compositions in the kit can be provided in a watertight or gas tight container which in some embodiments is substantially free of other components of the kit. For example, the collection device can be supplied in at least one container (e.g., the vial), and the sample transport media can be supplied in a container having sufficient reagent for a predetermined number of samples, e.g., 1, 2, 3 or greater. It is preferred that the components described herein are substantially pure and/or sterile.

In one embodiment, the informational material can include information about production of any of the components (e.g., collection devices, vials, sample transport media), concentration, date of expiration, batch or production site information, and so forth. In one embodiment, the informational material relates to methods for collecting samples using the components of the kit.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time. In some embodiments, the kit can be provided with components of various sizes, e.g., capillaries and caps of varying dimensions (e.g., any of the lengths described herein), such that a user can select the appropriate capillary and/or cap for particular applications.

Methods and Workflow of Using a Collection Device

In some embodiments, an example workflow using a collection device is described as below.

At step 1, a collection device including a capillary or absorbent paper and/or a cap is obtained. In some embodiments, the collection device is included in a kit. In some embodiments, the collection device can be delivered/obtained with the capillary or absorbent paper attached to the cap 120. In other embodiments, the collection device is delivered/obtained with the cap separate from the capillary or absorbent paper, e.g., before in use.

At step 2, optionally, the cap is connected firmly to the capillary or absorbent paper (e.g., from a distal end). The collection device can then be used to collect a sample (e.g., biological sample).

At step 3, the collection device, including the capillary and the cap, is sealed to the vial by screwing the threaded portion of the cap to the threaded portion of the vial. In some embodiments, the dimension and structure of the shortened collection device is configured to be fully and securely coupled to the opening of the vial.

At step 4, an automation device is used to remove the cap from the vial. [00345] At step 5, the collection device, including the cap operatively coupled to the incomplete capillary or absorbent paper, is detached from the vial after decapping.

In some embodiments, additional steps may include pricking or puncturing e.g., the dermis layer of the skin for the sample collection device to access e.g., the capillary beds that run through the subcutaneous layer of the skin.

In some embodiments, the workflow in the present disclosure further includes blood drying methods (e.g., for drying the blood in the cap or vial). In some embodiments, the workflow in the present disclosure further includes methods (e.g., surface treatment or other physical methods) to separate blood components.

Automation System and Process

In some embodiments, the collection device and/or the sample can be processed (e.g., after the collection device is deposited into a vial) using a manual process, a semi-automated process, or a fully automated (or automation) process. In some embodiments, an automation process includes using one or more automation devices. In some embodiments, the automation device includes a tube capper and decapper machine. In some embodiments, the automation device includes a liquid handling machine. In some embodiments, the automation device includes a shaker (e.g., an orbital shaker).

In general, the cap can have any structure that corresponds with any known or future developed automation device. For example, in some embodiments, the cap includes a hollow internal portion, an outer surface of the cap (e.g., top surface, circumferential surface, side surface), and/or one or more internal ribs, which enables the cap to interface with an automated device.

In some embodiments, an automation device can move, control, and/or manipulate at least a portion of the collection device after interfacing with the cap. In some embodiments, a portion of the automation device can extend into the hollow internal portion of the cap.

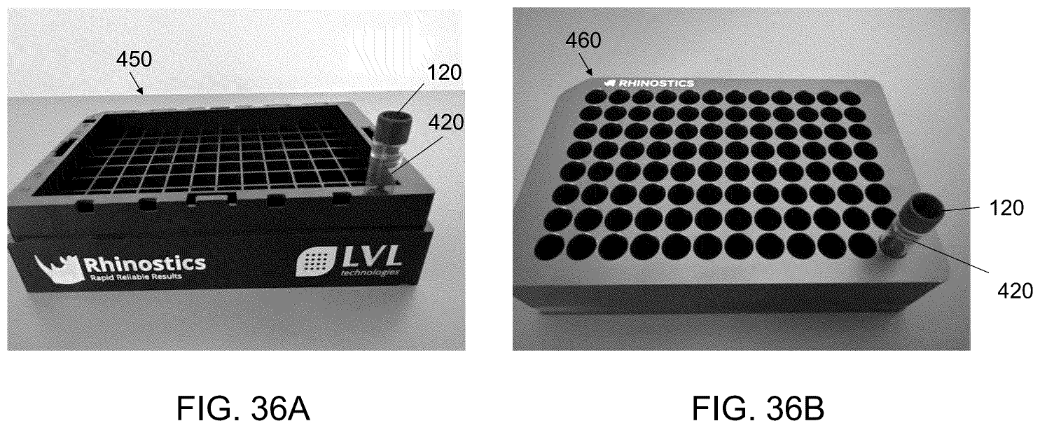

FIGS. 36A and 36B are views of embodiments of 96-well rack for storage of a collection device sealed to a vial 420, according to various embodiments. In some embodiments, the rack 450 includes one or more square wells (FIG. 36A). In some embodiments, the rack 460 includes one or more round wells (FIG. 36B).

In some embodiments, an automated method of processing a collection device includes using an automation system to perform at least one of the following steps: 1) receiving a collection device that has been contacted with a sample and deposited into a vial; 2) removing a cap from a vial; 3) removing at least a portion of a sample from the collection device and the vial (e.g., by removing a liquid within the vial and/or by removing the collection device); 4) transporting at least a portion of the sample to a testing location (as an alternative to steps 2) and 3) the sample can remain in the vial and a testing solution can be delivered into the vial); 5) testing at least a portion of the sample (e.g., to determine the presence of some substance) and capturing data resulting from the test; 6) further processing at least a portion of the sample.

In some embodiments, after receiving the collection device, a barcode (or a label) on the collection device and/or vial is detected using a barcode scanning machine. In some embodiments, an automation system for processing the collection device includes one or more devices (e.g., a tube capper, a decapper machine, a liquid handling machine, and/or a shaker). In some embodiments, the one or more devices are automated machines (e.g., robots).

In some embodiments, the step of removing at least a portion of a sample from the collection device and the vial further includes: removing the collection device from the vial (e.g., using the tube capper and decapper machine), adding a solution to the vial (e.g., using a liquid handling machine), placing the collection device back into the vial (e.g., using the tube capper and decapper machine), shaking the solution in the vial using a shaker in order to remove at least a portion of the sample from the incomplete capillary or absorbent paper of the collection device, removing the collection device from the vial and solution (e.g., using the tube capper and decapper machine, and/or removing a portion of the solution from the vial (e.g., using the liquid handling machine) for further processing (e.g., testing)

In some embodiments, the solution is saline, lysis buffer, PBS or other buffer.

In some embodiments, the further processing includes a diagnostic test. In some embodiments, the further processing includes nucleic acid (e.g., RNA or DNA) extraction, protein extraction, nucleic acid (e.g., RNA or DNA) amplification (e.g., PCR or isothermal amplification methods), and/or a detection assay (e.g., RT-qPCR). Non-limiting examples of isothermal amplification methods include: Recombinase Polymerase Amplification (RPA), Loop Mediated Isothermal Amplification (LAMP), Helicase-dependent isothermal DNA amplification (HDA), Rolling Circle Amplification (RCA), mass spectrometry, Nucleic acid sequence-based amplification (NASBA), strand displacement amplification (SDA), nicking enzyme amplification reaction (NEAR), and polymerase Spiral Reaction (PSR). In some embodiments, the tests can include antigen and/or protein-based tests. In some embodiments, the tests include analysis of immune or antibody status, vaccination status, presence or absence of protein biomarker, levels of protein biomarker, pathogen detection, and/or biological levels (e.g, Alc, hormone levels, or other biological levels). In some embodiments, the further processing includes mass spectrometry analysis or sequencing. In some embodiments, the further processing includes next generation sequencing and/or Sanger sequencing.

In some embodiments, the collection device described herein reduces the time of entire processes as described herein by in a range from about 1% to about 90%, about 1% to about 50%, about 5% to about 85%, about 10% to about 80%, about 15% to about 75%, about 20% to about 70%, about 25% to about 65%, about 30% to about 60%, about 35% to about 55%, or about 40% to about 50%, compared to a process using traditional methods (e.g., enzyme-linked immunoassay (ELISA), mass spectrometry, or other traditional methods to measure proteins and antibody levels).

In some embodiments, an automation method for processing a rack of one or more collection devices includes: 1) obtaining one or more vials, where the vial includes samples collected in a collection device and sealed to the vial using the cap (e.g., cap 120); 2) loading the vials (e.g., manually or by an automation process) to at least one rack (e.g., in a rack 450 or 460 in FIGS. 36A and 36B) for testing; 3) putting the rack of vials onto a robot for scanning (e.g., barcodes on the vials) e.g., in seconds; 4) passing the rack of vials to a handheld or fully automated decapping machine, where the automation device removes the caps (e.g., 96 caps) from the vials in the rack (e.g., in 30 seconds) and then moves the rack to a liquid handling robot (e.g., 30 seconds); 5) adding a saline solution (e.g., 100 uL in 10 seconds) using the liquid handling robot; 6) moving the rack back to the decapping robot e.g., from the liquid handling robot (e.g., 30 seconds); 7) replacing the caps (e.g., 30 seconds) of the vials; 8) moving the vials to e.g., an orbital shaker (e.g., 30 seconds), which shakes to move sample material into solution (e.g., 10 seconds); 9) moving the rack to the decapping robot (e.g., 30 seconds) and removing the caps (e.g., 30 seconds); 10) moving the rack back to the liquid handler (e.g., 30 second), which moves at least part of the sample into a microplate for e.g., downstream qPCR; 11) moving the rack back to the decapper robot, putting the caps back on; and 12) moving the rack to a storage location (e.g., 1.5 minutes). In some embodiments, the time for a portion or all the steps for processing a rack takes about 1, 2, 3, 4, 5, 6, 7, 8, 9, or 10 minutes.

### Additional Embodiments

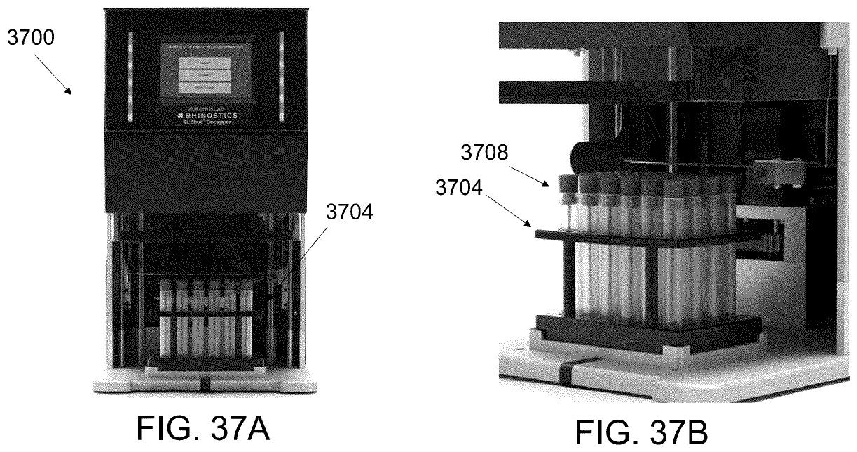

FIG. 37A is an image of an automated decapper 3700. The automated decapper 3700 may include any decapping robot, system, and/or process as described throughout this disclosure, without limitation. In some embodiments, the automated decapper 3700 may be configured to decap one or more collection devices, such as described above with reference to FIGS. 36A-B, without limitation. The automated decapper 3700 may include a rack 3704. The rack 3704 may be configured to securely position one or more collection devices, vials, and the like.

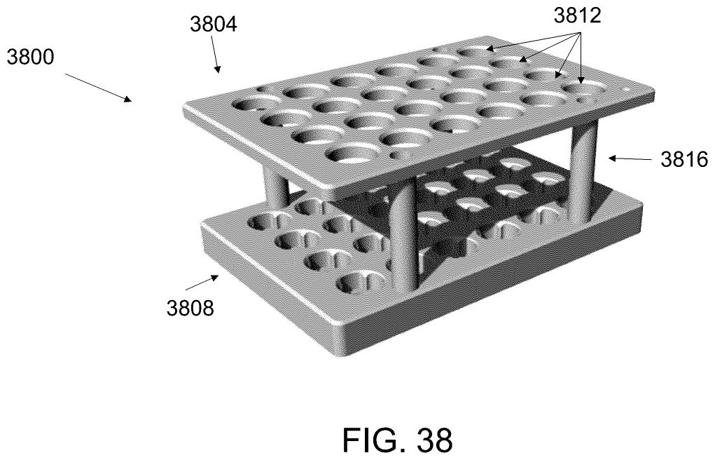

FIG. 37B is an image of the rack 3704. The rack 3704, in an embodiment, may have a 24 well rack format which may be referred to as a “Universal Format” rack. The rack 3704 may include any rack as described throughout this disclosure, without limitation. In some embodiments, the rack 3704 may be configured to securely hold 24 or more vials and/or collection devices 3708. In other embodiments, the rack 3704 may be configured to hold less than 24 vials and/or collection devices 3708. The rack 3704 may be as described below with reference to FIG. 38.

FIG. 38 illustrates a rack 3800 with a 24 well rack format. The rack 3800 may be made of, but not limited to, plastic, rubber, and/or other materials. In some embodiments, the rack 3800 may have a top portion 3804 and a bottom portion 3808. The top portion 3804 and the bottom portion 3808 may be similarly shaped and/or have similar dimensions. For instance, and without limitation, the top portion 3804 may have a length, width, and/or height that may match a corresponding length, width, or height of the bottom portion 3808. In some embodiments, the top portion 3804 and the bottom portion 3808 may differ in dimensions. For instance and without limitation, the bottom portion 3808 may have a greater height than a height of the top portion 3804. The bottom portion 3808 may have a greater volume than the top portion 3804 which may allow the bottom portion 3808 to act as a base, providing structural support to the top portion 3804. In some embodiments, the top portion 3804 and the bottom portion 3808 may have one or more holes 3812. In an embodiment, each hole 3812 of the top portion 3804 may correspond and/or be aligned with each hole 3812 of the bottom portion 3808. In a non-limiting example, the top portion 3804 and the bottom portion 3808 may have 24 holes 3812.

The rack 3800 may include one or more columns 3816. In some embodiments, the rack 3800 may include four columns 3816. The rack 3800 may have two or more columns on opposing sides of the bottom portion 3808. In some embodiments, the columns 3816 may have dimensions such as, but not limited to, circumference, diameter, length, height, and the like. The columns 3816 may have dimensions that may allow for structural support to be provided to the top portion 3804 through one or more columns 3816. For instance, and without limitation, the columns 3816 may have a circumference of about 10 cm. The columns 3816 may have a circumference greater or less than about 10 cm. In some embodiments, the columns 3816 may have a height that may allow a vial and/or collection device to fit inside a hole 3812 of the top portion 3804 and a hole 3812 of the bottom portion 3808. In some embodiments, a height of the columns 3818 may allow for a top of a vial placed in a hole of the top portion 3804 and the bottom portion 3808 to protrude from the rack 3800 which may allow for a decapping system to grasp the top of the vial.

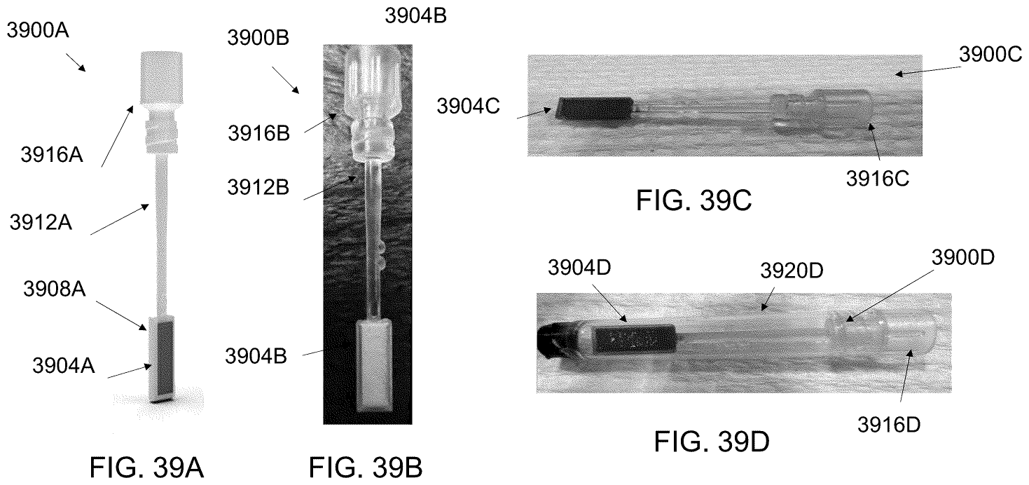

FIG. 39A illustrates a collection device 3900A with an absorbent insert 3904A. The collection device 3900A may include any collection device as described throughout this disclosure, without limitation. The collection device 3900A may include a collection end. A “collection end” as used in this disclosure is a structure formed to receive at least a biological sample. A collection end may include, but is not limited to, swabs, absorbent inserts/pads, capillaries, and/or other structures. Here, a collection end of the collection device 3900A is in the form of insert holder 3908A which may hold the absorbent insert 3904A. An “absorbent insert” as used in this disclosure is a material that collects fluid. The absorbent insert 3904A may be made of out, but not limited to, DBS paper, foam, cotton, glass fibers, and the like. The absorbent insert 3904 may be placed within an insert holder 3908A. The insert holder 3908A may be positioned at a bottom of the collection device 3900A. The insert holder 3908A may be configured to hold the absorbent insert 3904A. The absorbent insert 3904A may collect one or more fluids, such as, but not limited to, saliva, blood, mucus, and/or other fluids. The collection device 3900A may have a stem 3912A. The stem 3912A may be made from a same material as the rest of the collection device 3900A. In other embodiments, the stem 3912A may be made of a different material than the rest of collection device 3900A. The stem 3912A may connect a top portion of the collection device 3900A to the insert holder 3908A. For instance, the stem 3912A may have a first end and a second end. A first end of the stem 3912A may connect to the cap 3916A and a second end of the stem 3912A may connect to the collection end of the collection device 3900A, which is depicted here as the insert holder 3908A. The stem 3912A may be cylindrical, rectangular, or have other geometries. In some embodiments, the stem 3912A, collection device 3900A, and insert holder 3908A may be made out of a same mold from a manufacturing process. The collection device 3900A may include cap 3916A. The cap 3916A may include any cap as described throughout this disclosure, without limitation. The cap 3016A may be made of a same process and/or material of stem 3912A, insert holder 3908A, and/or the rest of the collection device 3900A. The cap 3916A may include one or more threads that may enable the cap 3916A to screw onto a receiving end of a vial, such as described below.

FIG. 39B is an image of a collection device 3900B with an absorbent insert 3904 B as described in FIG. 39A. The collection device 3900B is shown with an unused absorbent insert 3904B. The collection device 3900B may include a stem 3912B and/or cap 3916B, both of which may be the same as that of the stem 3912A and cap 3916A as described above with reference to FIG. 39A.

FIG. 39C is another image of the collection device 3900 as described above in FIG. 39A. The collection device 3900 is shown with a used absorbent insert 3904.

FIG. 39D is an image of the collection device 3900D as described above in FIG. 39A inside of a vial 3920D. The collection device 3900D is shown with a used absorbent insert 3904D. The collection device 3900D may act as a top to the vial 3912D through the cap 3916D. For instance and without limitation, the cap 3916D may be screwed onto a receiving end of the vial 3920D. The vial 3920D may have a screw-like structure that may allow the collection device 3900D to twist onto the vial 3920D. The vial 3920D may be made of plastic, glass, and/or other materials. In some embodiments, the vial 3920D may be clear which may allow a user or device to see the absorbent insert 3904D. In some embodiments, the vial 3920D may include a buffer or other chemical solution, such as any solution as described through this disclosure, without limitation.

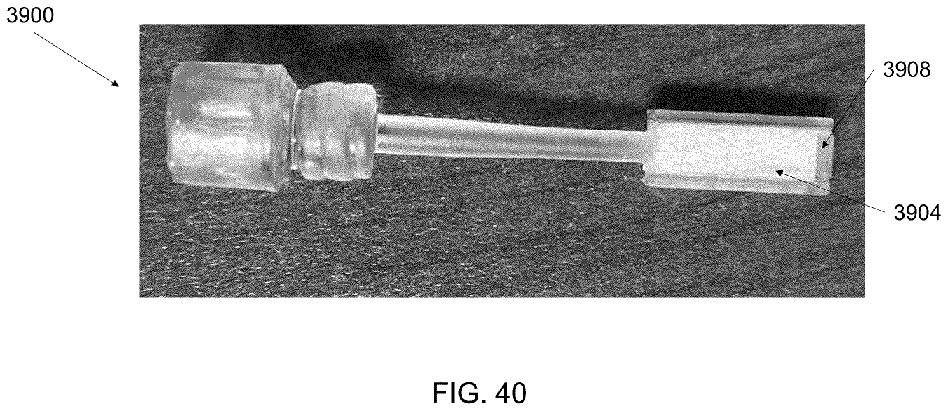

FIG. 40 is another image of the collection device 3900 as described above in FIG. 39A. The collection device 3900 includes the absorbent insert 3904. In some embodiments, the collection device 3900 may include an insert holder 3908. The insert holder 3908 may include one or more structures that allow for a secure positioning of the absorbent insert 3904 in the collection device 3904. For instance, in some embodiments, the insert holder 3908 may be injection molded. The insert holder 3908 may have one or more incisions, cuts, and the like. The insert holder 3908 may be injection molded to include an undercut that may enable a sliding of the absorbent insert 3904 into a secure position within the insert holder 3908. Undercuts may include cuts from a mold that leave behind grooves, hooks, and the like. In some embodiments, the absorbent insert 3904 may slide underneath two undercut sides of the insert holder 3908 and may stop at a bottom of the insert holder 3908 which may also have an undercut to hold the absorbent insert 3904 in place. Sides of the insert holder 3908 may be raised from a base of the insert holder 3908. For instance, sides of the insert holder 3908 may be raised about 2 mm from a base of the insert holder 3908. Sides of the insert holder 3908 may be raised greater than about 2 mm or less than about 2 mm, in some embodiments. The insert holder 3908 may include a bottom portion that may be raised above a base portion of the insert holder 3908. For instance, a bottom portion of the insert holder 3908 may be raised about 2 mm from a base of the insert holder 3908. A bottom portion of the insert holder 3908 may prevent the absorbent insert 3904 from falling out of the insert holder 3908. In some embodiments, the insert holder 3904 may be injection molded with one or more undercuts. Undercuts of the insert holder 3904 may be molded to hold the absorbent insert 3904.

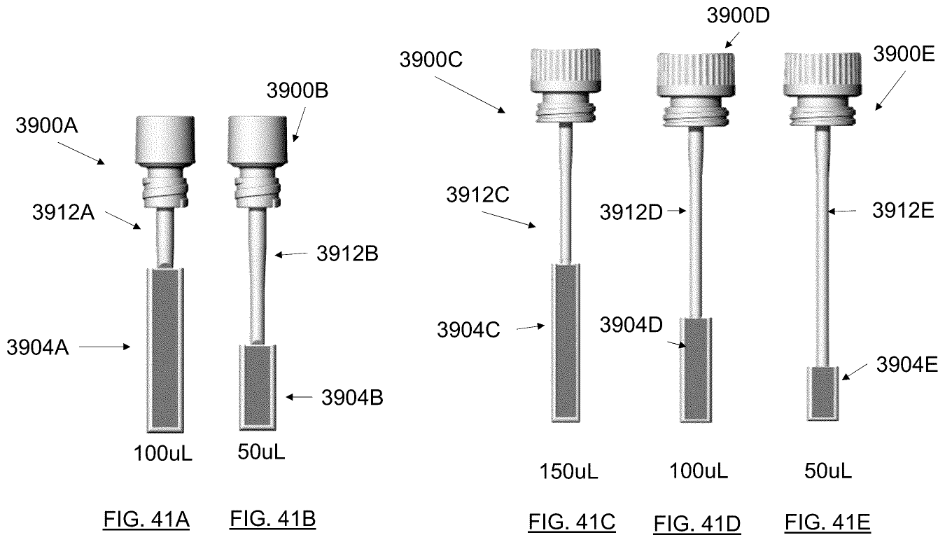

FIGS. 41A-E show various formats of a collection device with an absorbent insert. FIG. 41A is an illustration of a collection device 3900A with an absorbent insert 3904A, such as described above with reference to FIG. 39A. The collection device 3900A may have an absorbent insert 3904A that may have a fluid capacity. A fluid capacity of the absorbent insert 3904A may be between about 50 uL to about 200 uL. In some embodiments, a fluid capacity of the absorbent insert 3904A may be about 100 uL, 50 uL, or another volume amount. The stem 3912A of the collection device 3900A may be shorter than an original length and the absorbent insert 3904A may be longer than an original length of the collection device 3900A. A total length of the stem 3912A and the absorbent insert 3904A may remain the same, such as about 6 inches. A total length of the stem 3912A may be greater than or less than about 6 inches. A total length of the collection device 3900A may correspond to a well format. For instance, and without limitation, a well format may be a 96-well format. The collection device 3900A may have a longer absorbent insert 3904A with a fluid capacity of about 100 uL that may take up more of a total length of the collection device 3900A with the stem 3912A being a shorter length compared to the length of the absorbent insert 3904A.

FIG. 41B illustrates a collection device 3900B with an absorbent insert 3904B having a fluid capacity of about 50 uL. The absorbent insert 3904B may have a shorter length than the absorbent insert 3904A as shown in FIG. 41A which may be attributed to the absorbent inserts 3904B's smaller fluid capacity of 50 uL as compared to the 100 uL shown in FIG. 41A. The stem 3912B of the collection device 3900B may be longer than that of the stem 3912A shown in FIG. 41A which may be attributed to the smaller dimensions of the absorbent insert 3904B. A total length of the collection device 3900B may remain the same as compared to the collection device 3900A. For instance, the collection device 3900B may have a length corresponding to a 96-well format.

FIG. 41C illustrates a collection device 3900C with a universal large format. A universal large format may include about a 24-48 well format. The collection device 3900C may have a total length longer than that of the collection device 3900A shown in FIG. 41A. The absorbent insert 3904C may have a fluid capacity of about 150 uL. The absorbent insert 3904C may have greater dimensions that to allow for a greater fluid capacity of about 150 uL. Dimensions may include, without limitation, heights, widths, lengths, and the like.

FIG. 41D illustrates a collection device 3900D with a universal large format with the absorbent insert 3904D having a fluid capacity of about 100 uL. The absorbent insert 3904D may have smaller dimensions than the absorbent insert 3904C as shown in FIG. 41C due to its smaller fluid capacity. A total length of the collection device 3900D may remain the same as a total length of the collection device 3900C as shown in FIG. 41C

FIG. 41E illustrates a collection device 3900E in a universal or large format with an absorbent insert 3904E having a fluid capacity of about 50 uL. The absorbent insert 3904E may have smaller dimensions than the absorbent inserts shown in FIGS. 41C and 41D which may be due to the smaller fluid capacity of the absorbent insert 3904E. A total length of the collection device 3900E may be the same as a total length of the collection devices shown in FIGS. 41C-D which are also in a universal or large well format.

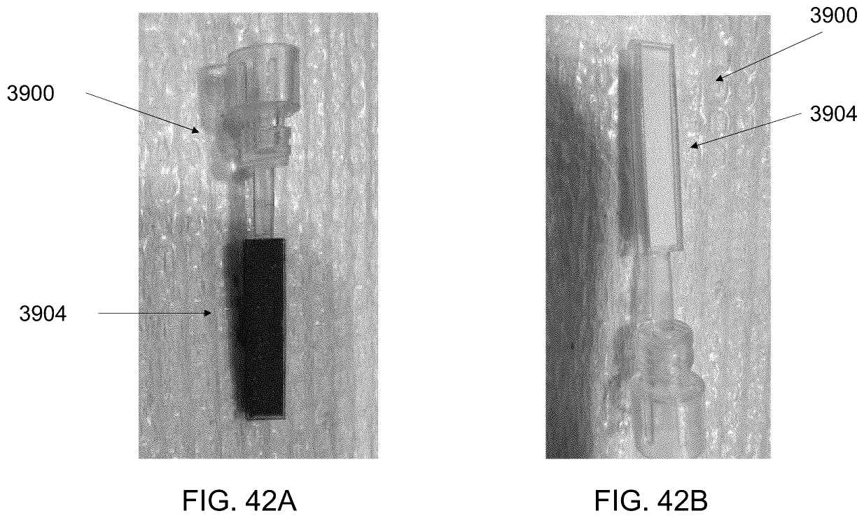

FIG. 42A is an image of the collection device 3900 with a used absorbent insert 3904 in a 96-well format with a fluid capacity of about 110 uL.

FIG. 42B is an image of the collection device 3900 with an unused absorbent insert 3904 in a 96-well format with a fluid capacity of about 110 uL.

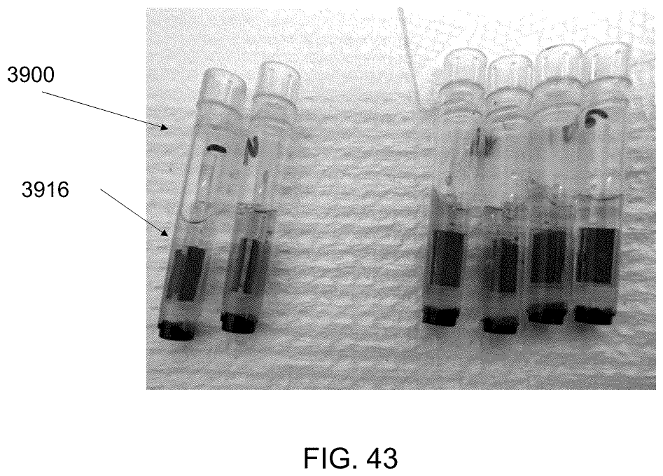

FIG. 43 is an image of various collection devices 3900 in vials 3916 with buffer solutions. Buffer solutions may include but are not limited to lysis buffers, PBS or other buffers.

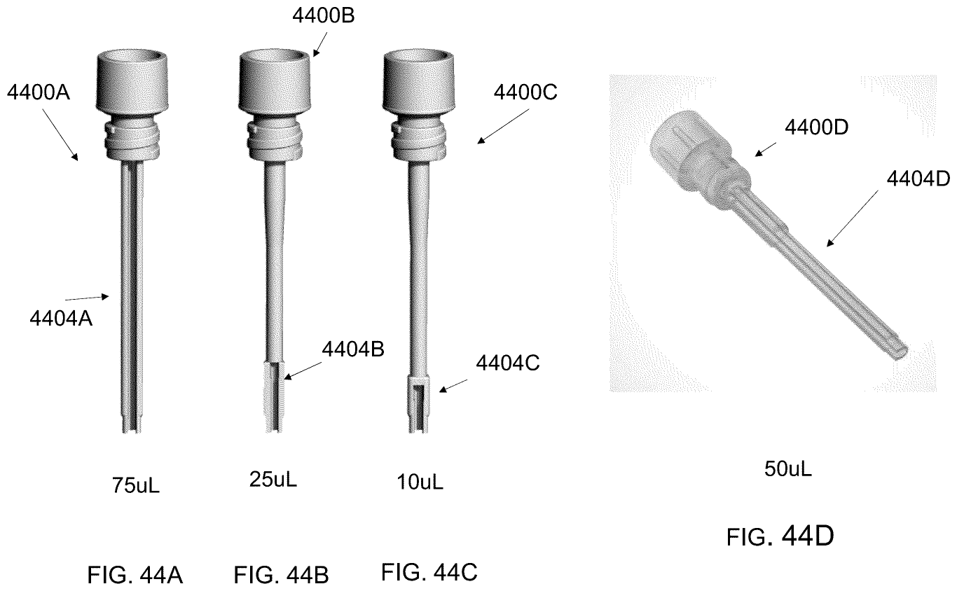

FIG. 44A illustrates a collection device 4400A with capillaries 4404A. The collection device 4400A may be the same as the collection device 3900 as described above with reference to FIG. 39 except with a replacement of the absorbent insert 3904 with the capillaries 4404A. In some embodiments, a collection end of the collection device 4400A may include capillaries 4404A. The capillaries 4404A may include one or more structures that may facilitate capillary action. The capillaries 4404A may have a fluid capacity of about 10 uL to about 100 uL. As shown in FIG. 44A, the capillaries 4404A have a fluid capacity of about 75 uL. The capillaries 4404A may include any capillaries as described throughout this disclosure without limitation.

FIG. 44B illustrates the collection device 4400B with capillaries 4404B having a fluid capacity of about 25 uL. The capillaries 4404B may be shorter than that of the capillaries 4404A as depicted in FIG. 44A due to the capillaries 4404B having a lower fluid capacity.

FIG. 44C illustrates a collection device 4400C with capillaries 4404C having a fluid capacity of about 10 uL. The capillaries 4404C may be shorter than the capillaries 4404A depicted in FIG. 44A due to the capillaries 4404C having a smaller fluid capacity.

FIG. 44D illustrates a collection device 4400D with capillaries 4404D having a fluid capacity of about 50 uL. The capillaries 4404D may be longer than the capillaries 4404A depicted in FIG. 44A due to the capillaries 4404D having a larger fluid capacity. Each collection device shown in FIGS. 44A-D may have a same total length which may be attributed to a 96-well format.

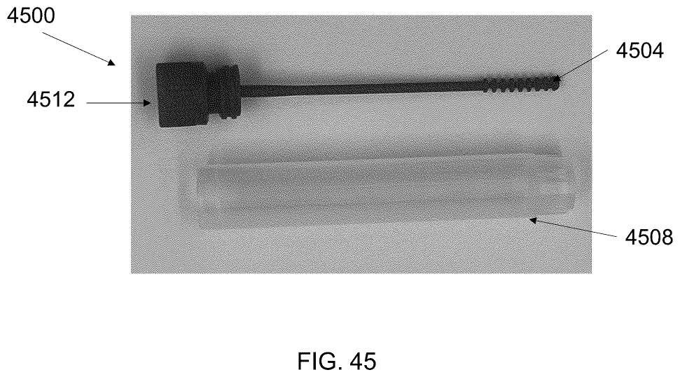

FIG. 45 is an image of a collection device 4500 in a universal size and/or large format. A universal size and/or large format may be applicable to, but not limited to, 12, 24, 36, 48, or other well rack layouts. The collection device 4500 may have swabbing end 4504. The swabbing end 4504 may have one or more textures, such as, but not limited to, rough, smooth, bumpy, and the like. The swabbing end 4054 may have a rippled design. A rippled design may help facilitate the collection of one or more fluids. In some embodiments, the swabbing end 4054 and the collection device 4500 may be made from a same mold, 3D printing process, and/or other manufacturing process. The vial 4508 may be configured to receive the collection device 4500. The vial 4508 may include any vial as described throughout this disclosure, without limitation. The collection device may have cap 4512. The cap 4512, the collection device 4500, and the swabbing end 4504 may all be made from a same mold and may be a uniform piece. For instance, and without limitation, the swabbing end 4504, cap 4512, and collection device 4500 may all be made of a same material, such as polypropylene or other materials. The cap 4512 may be configured to act as a cap of the vial 4508. For instance, the cap 4512 may screw onto a top portion of the vial 4508.

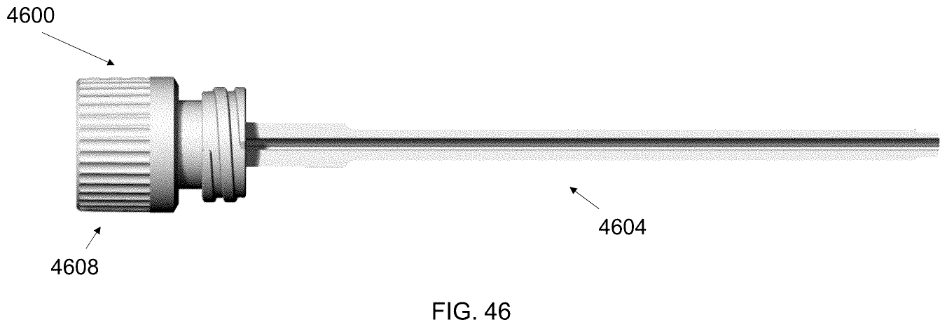

FIG. 46 illustrates a collection device 4600 with a high volume capillary 4604. The capillary 4604 may have a volume of about 50 uL or greater. The collection device 4600 and capillary 4604 may be in a universal or large format that may be suitable for 24-48 well formats. The collection device 4600 may have a cap 4608. The cap 4608 may be configured to screw into a vial, such as described above. The capillary 4604 may have a width of about 5 mm. In some embodiments, the capillary 4604 may have a width of greater than or less than about 5 mm.

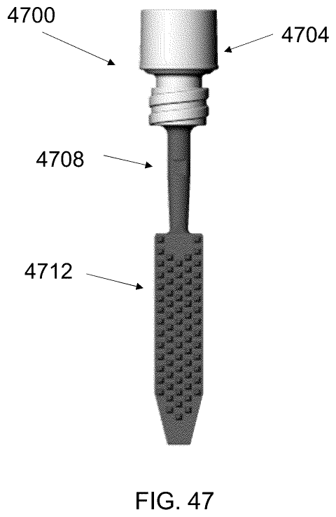

FIG. 47 illustrates a collection device 4700 with a collection tip 4712. In some embodiments, a collection end of collection device 4700 may include the collection tip 4712. The collection device 4712 may be injection molded and may have an automation compatible cap. The collection tip 4712 may be assembled into a cap 4704. The cap 4704 may be made from a different material than the collection tip 4712. In some embodiments, the collection tip 4712 may be made by. but not limited to, injection molding, 3D printing, compression molding, urethane casting, blow molding, CNC machining, and/or other manufacturing methods. A manufacturing process of the collection tip 4712 may be different than that of the rest of the collection device 4700, such as the cap 4074. As a non-limiting example, the cap 4704 may be manufactured through compression molding while the collection tip 4712 may be manufactured by 3D printing. Various manufacturing methods may be selected based on utility of the collection device 4700. For instance and without limitation, the cap 4704 may be manufactured by injection molding to fit a specific well format while the collection tip 4712 may be manufactured by 3D printing to have a rough surface texture that may allow for a higher collection of fluids, such as mucus. The collection tip 4712 may have a surface texture, such as rigid, smooth, bumpy, rough, and/or other textures. Surface textures of the collection tip 4712 may be selected for various fluids, such as, but not limited to, mucus, blood, saliva, and/or other fluids.

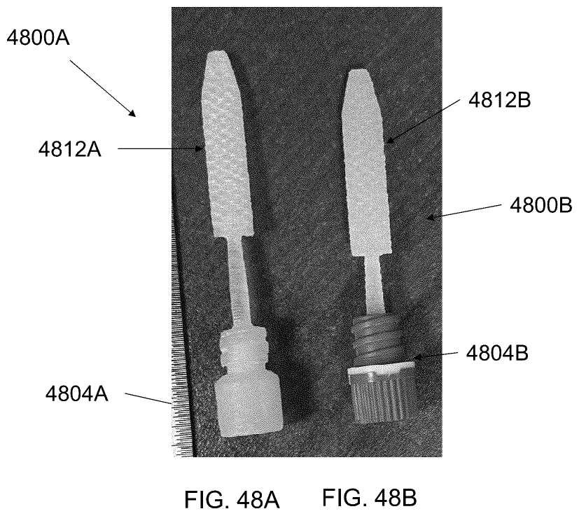

FIG. 48A is an image of a collection device 4800A with collection tip 4812A and cap 4804A. The collection device 4800A, the cap 4804A, and/or the collection tip 4812A may be 3D printed as a single unit. In some embodiments, the collection device 4800A, the cap 4804A, and/or the collection tip 4812A may be made from SLS polypropylene.

FIG. 48B is another image of a collection device 4800B with collection tip 4812B and cap 4804B. The collection device 4800B and/or the collection tip 4812B may be 3D printed. The cap 4804B may be injection molded and/or manufactured by a different process than the collection tip 4812B. The collection tip 4812B may be assembled into cap 4804B. The cap 4804B and the collection tip 4812B may be joined by heat staking or other methods. In some embodiments, the cap 4804B may be automation compatible. For instance, the cap 4804B may be compatible with one or more automation processes as described through this disclosure, without limitation.

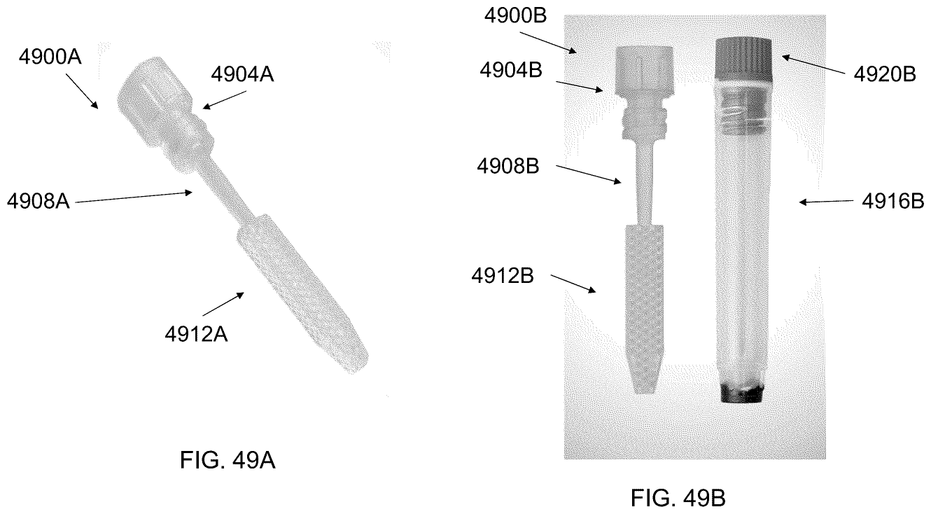

FIG. 49A illustrates a collection device 4900. The collection device 4900A may have collection tip 4912A, stem 4908A, and/or cap 4904A. The collection device 4900A may be as described above with reference to FIG. 47, without limitation.

FIG. 49B illustrates the collection device 4900B described in FIG. 49A adjacent to vial 4916B. The vial 4916B may include any vial as described throughout this disclosure, without limitation. The vial 4916B may act as a transport tube. For instance, the collection device 4900B may be placed inside of an interior of the vial 4916B. The vial 4916B may be configured to receive the collection device 4900B. In some embodiments, the vial 4916B may include a vial cap 4920B. The vial cap 4920B may be configured to mate with a top portion of the collection device 4900B, such as through a screw-like rotation or other processes. In other embodiments, the vial cap 4920B may be heat staked with a top portion of the collection device 4900B, such as the cap 4904B.

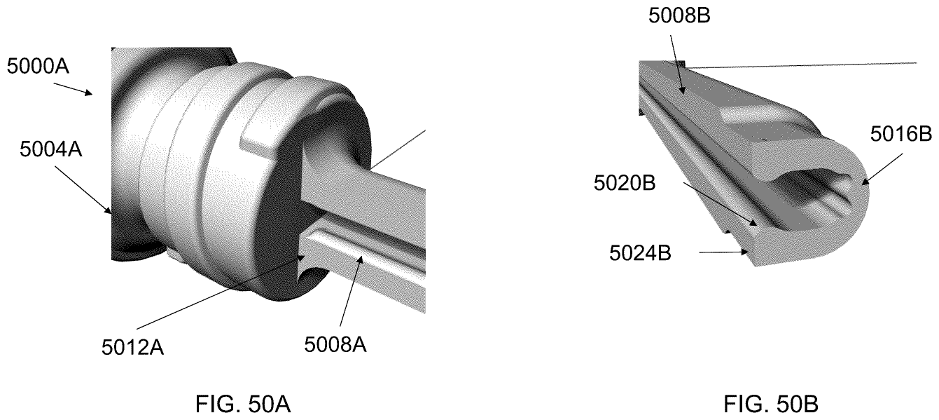

FIG. 50A illustrates a design for de-molding of the collection device 5000A. A top of the collection device 5000A, such as the cap 5004A may have an intersection 5012A between itself and the capillaries 5008A. A mold of the collection device 5000A may have an overhang which may stop before the intersection 5012A. An overhang of a mold of the collection device 5000A may allow a material of the collection device 5000A to flex and/or release from the mold.

FIG. 50B illustrates a design for capillaries 5008B. The capillaries 5008B may include an outer surface 5016B. The outer surface 5016B may include an interior portion 5020B and an exterior portion 5024B. The capillaries 5008B may be part of the interior portion 5020B. The inner portion 5020B may form an overhang. An overhang of the inner portion 5020B may include the inner portion 5020B extending towards a center of the capillaries 5008B. An overhang of the inner portion 5020B may allow for improved capillary action. An overhang of the inner portion 5020B may extend about 2 mm inwards from the exterior portion 5024B. In some embodiments, an overhang of the inner portion 5020B may extend greater than or less than about 2 mm from the exterior portion 5024B.

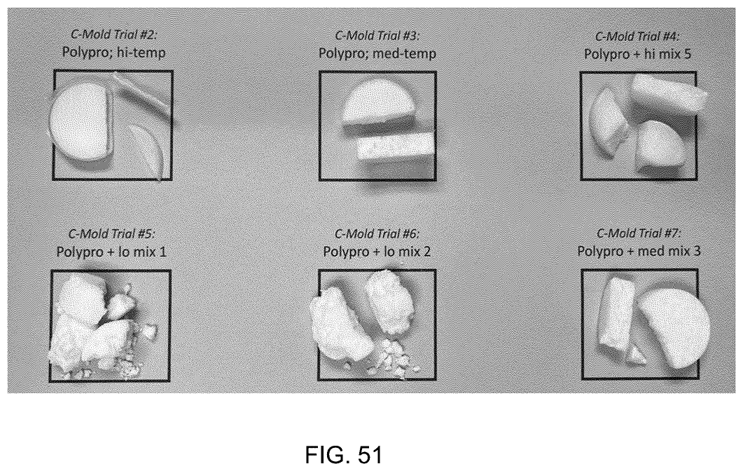

FIG. 51 illustrates various compression molding of powders. The powders may be used for 3D printing. In trial 2, polypropylene was compressed at a high temperature. A high temperature may include a temperature of about 400 degrees Celsius. In some embodiments, a high temperature may be greater than or less than about 400 degrees Celsius. The resulting compression mold had strong structural integrity. In trail 3, a polypropylene power was compressed at medium temperature. A medium temperature may include a temperature of about 200 degrees Celsius. In some embodiments, a medium temperature may include a temperature greater than or less than about 200 degrees Celsius. In trail 4, a polypropylene powder was compressed with a high temperature with a mix of materials. In trail 5, a polypropylene powder was compressed with a low temperature with a different mix of materials than in trail 5. In trail 6, a polypropylene powder was compressed with a low temperature with a different mix of materials than in trials 4 and 5. In trail 7, a polypropylene powder was compressed with a medium temperature with a mix of materials different than in trails 4-6. Different mixes of materials may include different ratios of one or more materials to one or more other materials. Ratios may include, without limitation, anywhere between 1:100 to about 100:1 of one material to another material. Materials may include, but are not limited to, thermoset resins, thermoplastic resins, and/or other materials. Materials may include any materials as described throughout this disclosure, without limitation.

### Definitions

The term “a” or “an” refers to one or to more than one (i.e. at least one) of the grammatical objects of the article. By way of example “an analogue” means one analogue or more than one analogue.

The terms “about” or “substantially” that modify a condition or relationship characteristic of a feature or features of an embodiment of the invention, are to be understood to mean that the condition or characteristic is defined to within tolerances that are acceptable for operation of the embodiment for an application for which it is intended. For example, the terms “about,” “substantially,” and/or “close” with respect to a magnitude or a numerical value may imply to be within an inclusive range of −10% to +10% of the respective magnitude or value.

The term “overmolding” refers to a process wherein a single part is created using two or more different materials in combination. For example, the first material, sometimes referred to as the substrate, is partially or fully covered by subsequent materials (i.e., overmold materials) during the manufacturing process. The term “subject,” “individual,” or “body,” refers to a human, an animal.

The term “complete capillary” or “closed capillary” refers to a capillary that includes a hollow interior that is entirely surrounded by the inner wall of the capillary. A cross-section of a complete capillary may include a closed shape whose line segments and/or curves form a closed region.

The term “incomplete capillary” or “open capillary” refers to a capillary including a cross-section of an open shape whose line segments and/or curves do not meet one or more endpoints of one side.

The term “diameter” refers to the distance of a straight line passing through the axial center of a circular cross section. A diameter as described herein can include any one of a maximum, minimum, average, or medium diameter.

The term “automated” or “automation” refers to a device, system, or method that can be achieved using automatic means that are independent of human operators or supervision.

The term “automation compatible” refers to having features adapted to interact with any equipment that facilitates performance of a test or assay (e.g., a handheld or fully automated decapping machine, handheld decapper equipment, liquid handlers, laboratory robotics, tube cappers, transport robotics, shaking apparatus, liquid transport mechanism, etc.).

The term “sample collection device” or “collection device” refers to a device that can be used to collect biological samples. The term “sample collection structure,” “collection structure,” or “sample collection vehicle” refers to a structure or element that can be used to collect biological samples.

The term “sample,” “biological sample” or similar terms can include biological fluids (e.g., blood) at any biological location (e.g., finger, arms, legs, or other suitable regions) of a subject (e.g., a human, an animal, etc.), and/or any biological tissues (e.g. buccal cells, sores, poxes, tissues, tumors or pathogens.

The term “channel” refers to a portion of a capillary where the fluids are collected, stored, conveyed through, or transported. In some embodiments, the term “channel” refers to the “cavities” as shown in FIGS. 8-11. In some embodiments, a collection device can have 1, 2, 3, 4, 5, 6, or more channels.

Each numerical value presented herein is contemplated to represent a minimum value or a maximum value in a range for a corresponding parameter. Accordingly, when added to the claims, the numerical value provides express support for claiming the range, which may lie above or below the numerical value, in accordance with the teachings herein. Every value between the minimum value and the maximum value within each numerical range presented herein (including in the figures), is contemplated and expressly supported herein, subject to the number of significant digits expressed in each particular range. Absent express inclusion in the claims, each numerical value presented herein is not to be considered limiting in any regard.

Having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive. The terms and expressions employed herein are used as terms and expressions of description and not of limitation and there is no intention, in the use of such terms and expressions, of excluding any equivalents of the features shown and described or portions thereof. The structural features and functions of the various embodiments may be arranged in various combinations and permutations, and all are considered to be within the scope of the disclosed invention. Unless otherwise necessitated, recited steps in the various methods may be performed in any order and certain steps may be performed substantially simultaneously.

1. A tissue collection device, comprising:
a cap adapted to interface with an automated device;
a stem having a first end and a second end, the stem mated with the cap at the first end; and
a collection tip positioned distal from the cap and mated to the second end of the stem, the collection tip adapted to retrieve a tissue sample of a patient.

2. The tissue collection device of claim 1, wherein the cap comprises an external threaded portion adapted to mate with a vial.

3. The tissue collection device of claim 1, wherein the cap comprises a hollow cylinder with at least one internal groove or at least one internal ridge.

4. The tissue collection device of claim 1, wherein the cap and collection tip are manufactured from the same mold in an injection molding process.

5. The tissue collection device of claim 1, wherein the cap and collection tip are manufactured separately and joined together through a heat staking process.

6. The tissue collection device of claim 1, wherein the collection tip comprises one of fin or bump structures or a combination thereof.

7. The tissue collection device of claim 1, wherein the collection tip comprises cavities formed to collect and hold cells of the tissue sample.

8. The tissue collection device of claim 1, wherein the collection tip comprises a porous structure.

9. The tissue collection device of claim 1, wherein the automated device comprises a tube capper and decapper machine.

10. The tissue collection device of claim 1, wherein the tissue sample includes one of human cheek, nose, or throat cells.

11. A method of collecting a tissue sample, the method comprising the steps of:
retrieving a tissue sample using the tissue collection device of claim 1.
